(19) United States
(12) Patent Application Publication
(10) Pub. No.: US 2010/0280056 A1
(43) Pub. Date: Nov. 4, 2010

(54) IDENTIFICATION OF SUBJECTS LIKELY TO BENEFIT FROM STATIN THERAPY

(75) Inventors: Levy Kopelovich, Annandale, VA (US); Steven M. Lipkin, Irvine, CA (US); Gad Rennert, Haifa (IL); Stephen B. Gruber, Ann Arbor, MI (US); Victor Moreno, Barcelona (ES)

Correspondence Address: KLARQUIST SPARKMAN, LLP (OTT-NIH), 121 S.W. SALMON STREET, SUITE #1600, PORTLAND, OR, 97204-2988, US

(73) Assignees: The Govt. of the U. S. as Represented by the Secretary of the Dept. of Health and Human Svcs.; The Regents of the University of California; Mor Research Applications; The Regents of the University of Michigan

(21) Appl. No.: 12/741,332

(22) PCT Filed: Nov. 4, 2008
(86) PCT No.: PCT/US08/82359

§ 371 Date: May 4, 2010

Related U.S. Application Data
(60) Provisional application No. 60/985,587, filed on Nov. 5, 2007.

(51) Int. Cl. A61K 31/505; C12Q 1/68; A61K 31/351; A61K 31/225; A61K 31/40; A61P 35/00

(52) U.S. Cl. 514/275; 435/6; 514/460; 514/548; 514/423

(57) ABSTRACT

Methods are provided herein to determine if a subject is a candidate for treatment with an inhibitor of 3-hydroxy-3-methylglutaryl coenzyme A reductase (HMGCR). The method includes determining the presence of at least one polymorphism in the HMGCR gene in a sample from a subject. The presence of at least one polymorphism indicates that the subject is a candidate for treatment with a statin, for example to decrease risk of or treat cancer, cardiovascular disease, diabetes, obesity, inflammatory disease, or auto-immune disease.

### CROSS REFERENCE TO RELATED APPLICATION

This application claims the benefit of U.S. Provisional Application No. 60/985,587, filed Nov. 5, 2007, which is incorporated herein in its entirety.

### FIELD OF THE DISCLOSURE

This disclosure relates to the field of individualized medicine, specifically to the identification of subjects for treatment with an inhibitor of 3-hydroxy-3-methylglutaryl coenzyme A reductase.

### BACKGROUND

Inhibitors of 3-hydroxy-3-methylglutaryl coenzyme A reductase (HMGCR), which are also known as statins, are widely prescribed drugs. Statins lower low-density lipoprotein (LDL) cholesterol levels and reduce the risk of cardiovascular disease. In addition to their cholesterol-lowering effects, statins have anti-inflammatory effects, and have been shown to reduce levels of markers of inflammation, such as C-reactive protein (Kleeman et al., Blood 103: 4188, 2004).

Statins specifically inhibit the rate-limiting step of the mevalonate pathway, an effect that influences cholesterol homeostasis and other diverse cellular functions. New evidence suggests that atherosclerosis and cancer have similar underlying molecular mechanisms, both having lipid abnormalities and a pro-inflammatory phenotype. Like nonsteroidal anti-inflammatory agents, statins target lipid metabolism, have significant anti-inflammatory effects, and can influence cardiovascular mortality (see Katz, Natl. Clin. Pract. Oncol. 2:82-89, 2005).

The relationship between statin use and cancer risk has been evaluated in numerous observational studies and as a secondary outcome in randomized controlled trials evaluating the effects of statins on cardiovascular outcomes. Although there are plausible biologic mechanisms to suggest that statins could inhibit cellular proliferation, the epidemiologic data on reduction in cancer risk among statin users are variable (Moorman and Hamilton, Epidemiology 18:194-6, 2007). Several clinical trials have also evaluated statins as a potential anti-cancer therapy, in combination with other chemotherapy agents. Some clinical studies showed promising results, while others showed no beneficial effect of statin therapy (Hindler et al., Oncologist 11:306-315, 2006). Thus, there is a need for methods to identify individuals who would benefit most from treatment with statins.

### SUMMARY

Polymorphisms in HMGCR are disclosed herein that are of use for identifying subjects that can be treated with statins. Methods are disclosed herein for identifying a subject as a candidate likely to benefit from treatment with an inhibitor of HMGCR. In some embodiments, the method includes identifying candidates for treatment with an inhibitor of HMGCR to decrease risk of developing cancer or to treat cancer. In several embodiments, the cancer is colorectal cancer, melanoma, breast cancer, prostate cancer, or lung cancer. In additional embodiments, the inhibitor of HMGCR is simvastatin, pravastatin, rosuvastatin, or atorvastatin.

In additional embodiments, the method includes identifying candidates for treatment with an inhibitor of HMGCR to decrease risk of developing or to treat cardiovascular disease, diabetes, obesity, inflammatory disease, and/or auto-immune disorders.

In several embodiments, the method includes detecting the presence of at least one polymorphism in an HMGCR gene in a sample from the subject, wherein the presence of at least one polymorphism indicates that the subject is a candidate for treatment with an HMGCR inhibitor. In one embodiment, the method includes detecting the presence of a polymorphism having a G at nucleotide position 224 of intron 5 of an HMGCR gene. In a further embodiment, the method includes detecting the presence of a polymorphism having an A at nucleotide position 1176 of intron 11 of an HMGCR gene. In another embodiment, the method includes detecting the presence of a polymorphism having a T at nucleotide 372 downstream of the termination codon of an HMGCR gene. In a further embodiment, the method includes detecting the presence of a polymorphism having an A at nucleotide position 45 of intron 13 of an HMGCR gene. Additional embodiments include detecting the presence of more than one HMGCR polymorphism in combination. In a particular embodiment, the method includes detecting the presence of at least one polymorphism in an inhibitor binding domain of an HMGCR gene.

In one example, the method for identifying a candidate for treatment with an inhibitor of HMGCR to decrease the risk of cancer further includes determining a level of a C-reactive protein (CRP) in a sample from a subject, wherein an elevated level of CRP as compared to a control identifies a candidate for treatment with an HMGCR inhibitor.

Additional embodiments include kits for identifying a subject as a candidate for treatment with an inhibitor of HMGCR. In one example, the kit includes primers or probes to determine the presence of at least one polymorphism of the HMGCR gene. In several embodiments, the kit includes primers or probes that hybridize to an HMGCR gene nucleic acid sequence.

The foregoing and other objects, features, and advantages will become more apparent from the following detailed description, which proceeds with reference to the accompanying figures.

### BRIEF DESCRIPTION OF THE DRAWINGS

FIG. 1 is a haplotype plot of HMGCR and SNPs in linkage disequilibrium with rs12654264.

FIG. 2 is a photograph of a gel showing the presence of full length HMGCR and alternatively spliced HMGCR (HMGCRv1) in several colon cancer cell lines.

FIG. 3 is a graph showing total cholesterol content in colon cancer cell lines with different rs12654264 genotypes with and without treatment with atorvastatin. The left panel shows the data grouped by rs12654264 genotype (AA, AT, and TT). The right panel shows the data grouped by presence or absence of the rs12654264 TT genotype (TT or AT/AA).

FIG. 4 is a graph showing the change in cholesterol levels in atorvastatin treated colon cancer cell lines with different rs12654264 genotypes. The left panel shows the data grouped by rs12654264 genotype (AA, AT, and TT). The right panel shows the data grouped by presence or absence of the rs12654264 TT genotype (TT or AT/AA).

FIG. 5 is a graph showing the atorvastatin-dependent change in the ratio of HMGCRv1 to HMGCR transcripts in colon cancer cell lines with different rs12654264 genotypes. The left panel shows the data grouped by rs12654264 genotype (AA, AT, and TT). The right panel shows the data grouped by presence or absence of the rs12654264 TT genotype (TT or AT/AA).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequences are listed in Appendix I. In the accompanying sequence listing:

SEQ ID NO: 1 is the nucleic acid sequence of an exemplary human HMGCR gene.

SEQ ID NOs: 2 and 3 are the nucleic acid and amino acid sequences of an exemplary human of human HMGCR.

SEQ ID NO: 4 is a portion of a genomic sequence of human HMGCR which includes the polymorphism at position 224 of intron 5 and the 50 nucleotides flanking each side of the polymorphism.

SEQ ID NO: 5 is a portion of a genomic sequence of human HMGCR which includes the polymorphism at position 1176 of intron 11 and the 50 nucleotides flanking each side of the polymorphism.

SEQ ID NO: 6 is a portion of a genomic sequence of human HMGCR which includes the polymorphism at position 372 downstream of the termination codon and the 50 nucleotides flanking each side of the polymorphism.

SEQ ID NO: 7 is a portion of a genomic sequence of human HMGCR which includes the polymorphism at position 45 of intron 13 and the 50 nucleotides flanking each side of the polymorphism.

SEQ ID NOs: 8-28 are synthetic oligonucleotides for amplification of HMGCR variants.

### DETAILED DESCRIPTION

### I. Abbreviations

3′ UTR: 3′ untranslated region

ASO: allele-specific oligonucleotide

CRC: colorectal cancer

CRP: C-reactive protein

HMG-CoA: 3-hydroxy-3-methylglutaryl coenzyme A

HMGCR: 3-hydroxy-3-methylglutaryl coenzyme A reductase gene or protein

htSNP: haplotype tagging single nucleotide polymorphism

LDL: low-density lipoprotein

LSO: locus-specific oligonucleotide

PCR: polymerase chain reaction

SNP: single nucleotide polymorphism

WGA: whole genome amplification

### II. Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “a probe” includes single or plural probes and can be considered equivalent to the phrase “at least one probe.”

As used herein, the term “comprises” means “includes.” Thus, “comprising a probe” means “including a probe” without excluding other elements. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the invention, the following explanations of terms are provided:

Amplification: To increase the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample, for example the number of copies of an HMGCR nucleic acid, such as a polymorphic HMGCR nucleic acid, for example an HMGCR nucleic acid in which the nucleotide at position 224 of intron 5 is a G, an HMGCR nucleic acid in which the nucleotide at position 1176 of intron 11 is an A, an HMGCR nucleic acid in which the nucleotide 372 bases downstream of the termination codon is a T, or an HMGCR nucleic acid in which the nucleotide at position 45 of intron 13 is an A. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881); repair chain reaction amplification (see PCT Publication No. WO 90/01069); ligase chain reaction amplification (see European patent publication No. EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134), amongst others.

Ashkenazi Jew: A person who has Jewish ancestors from Central or Eastern Europe, including for example, Germany, Austria, Poland, Lithuania, and Russia. Approximately eighty percent of American Jews are of Ashkenazi descent. Particular genetic diseases are more common among Ashkenazi Jews than among other populations, such as Tay-Sachs disease, Bloom syndrome, Canavan disease, cystic fibrosis, Fanconi anemia, Gaucher disease and Niemann-Pick disease. Rates of colorectal cancer are also disproportionately high, and particular mutations in the breast cancer genes BRCA1 and BRCA2 are more common in Ashkenazi Jews than in other populations.

Auto-immune disorder: A disorder in which the immune system produces an immune response (e.g. a B cell or a T cell response) against an endogenous antigen, with consequent injury to tissues. Exemplary autoimmune diseases affecting mammals include rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (e.g., Crohn's disease, ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosus, autoimmune thrombocytopenia purpura, Goodpasture's syndrome, Addison's disease, systemic sclerosis, polymyositis, dermatomyositis, autoimmune hemolytic anemia, pernicious anemia, and the like.

C-Reactive Protein: C-reactive protein (CRP) is a plasma protein which is a marker of systemic inflammation. For example, CRP levels are elevated in acute infection, lupus erythematosus, rheumatoid arthritis, and inflammatory bowel disease. CRP levels have also been shown to be elevated in individuals with acute ischemia or myocardial infarction, and have been associated with increased risk of cardiovascular disease (see, e.g. U.S. Patent Application No. 2006/0115903, incorporated herein by reference). Individuals with CRP levels of less than 1 mg/L are considered to be at low risk, CRP levels between 1 mg/L and 3 mg/L are considered to be at moderate risk, and CRP levels of >3 mg/L are considered to be at high risk for cardiovascular disease (Pearson et al. Circulation 107:499-511, 2003). The amino acid sequence of CRP is known for several mammalian species (see, for example, Taylor et al., Biochem. J. 221: 903-6, 1984; GENBANK® Accession No. CAA39671, May 27, 1992, incorporated herein by reference).

Some studies have also identified CRP as a potential marker for increased cancer risk (see e.g., Mazhar and Ngan, Q. J. Med. 99:555-559, 2006). In these studies, individuals with cancer had higher levels of CRP than individuals without cancer.

Cardiovascular disease: Disorders related to the cardiovascular system, such as, but not limited to, artherosclerosis, coronary artery disease, myocardial ischemia and infarction, intermittent claudication, bowel ischemia, retinal ischemia, transient ischemic attacks, ischemic strokes, renal artery stenosis, and other conditions associated with cardiovascular dysfunction.

Decrease: Becoming less or smaller, as in number, amount, size, or intensity. In one example, decreasing the risk of a disease (such as cancer, cardiovascular disease, diabetes, obesity, inflammatory disease, or auto-immune disorder) includes a decrease in the likelihood of developing the disease by at least about 20%, for example by at least about 30%, 40%, 50%, 60%, 70%, 80%, or 90%. In another example, decreasing the risk of a disease includes a delay in the development of the disease, for example a delay of at least about six months, such as about one year, such as about two years, about five years, or about ten years.

In one example, decreasing the signs and symptoms of cancer includes decreasing the size, volume, or number of tumors (such as colorectal tumors) or metastases by a desired amount, for example by at least 5%, at least 10%, at least 15%, at least 20%, at least 25%, at least 30%, at least 50%, at least 75%, or even at least 90%, as compared to a response in the absence of the therapeutic composition.

Diabetes: Diabetes mellitus is the most common of the serious metabolic diseases affecting humans. It may be defined as a state of chronic hyperglycemia, i.e. excess sugar in the blood, consequent upon a relative or absolute lack of insulin action.

Diabetes mellitus is classified into two major forms; Type 1 and Type 2 diabetes. Type 1 diabetes, also referred to as insulin-dependent diabetes (IDDM), is an autoimmune disease which is associated with almost complete loss of the insulin-producing pancreatic β-cells. This loss of β-cells results in life-long insulin dependence. Type 1 diabetes can occur at any age, and it has been estimated that about 1% of all newborns will develop this disease during their lifetime. Type 2 diabetes or non-insulin dependent diabetes (NIDDM) refers to a group of disorders characterized by high blood levels of glucose (hyperglycemia) and a resistance to insulin, and occurs in patients with impaired pancreatic β-cell function. The absence of insulin in patients with Type 1 diabetes and the insulin resistance in Type 2 diabetes results in decreased absorption of sugar from the bloodstream, and hence excess sugar accumulates in the blood. Both types of diabetes are associated with shortened life expectancy, and with significant morbidity, such as vascular disease, blindness and atherosclerosis.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal (termination codon). The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes HMGCR, or a fragment thereof, encompasses both the sense strand and its reverse complement. Thus, for instance, it is appropriate to generate probes or primers from the reverse complement sequence of the disclosed nucleic acid molecules.

Genomic target sequence: A sequence of nucleotides located in a particular region in the human genome that corresponds to one or more specific genetic abnormalities, such as a nucleotide polymorphism, a deletion, an insertion, or an amplification. The target can be for instance a coding sequence; it can also be the non-coding strand that corresponds to a coding sequence. The target can also be a non-coding sequence, such as intronic sequence. In one example, a genomic target sequence is a genomic sequence of a gene that encodes an HMGCR protein, or portion thereof.

Haplotype: The ordered, linear combination of polymorphisms (e.g., single nucleotide polymorphisms, SNPs) in the sequence of each form of a gene (on individual chromosomes) that exists in a population.

HMGCR: 3-hydroxy-3-methylglutaryl coenzyme A reductase. HMGCR catalyzes the NADP-dependent conversion of HMG-CoA to mevalonate in the rate-limiting step of isoprenoid biosynthesis, which includes the synthesis of cholesterol. HMGCR is the direct and specific target of the statin family of cholesterol lowering drugs.

Heme A, ubiquinone, and dolichol are also products of the mevalonate pathway. This pathway is also responsible for production of prenylated (farnesylated or geranyl-geranylated) proteins, such as the Ras/Rho family of proteins. Further, cholesterol is the precursor of all steroid hormones, such as testosterone, estradiol, glucocorticoids, and mineral corticoids. HMGCR activity is tightly regulated by transcriptional, post-transcriptional, and post-translational mechanisms. It is regulated by a negative feedback mechanism mediated by sterols and non-sterol metabolites derived from mevalonate. HMGCR is widely expressed throughout the body.

The human HMGCR gene is located on chromosome 5q13.3 and comprises twenty exons, which span approximately 25 kb (GENBANK® Accession No. NC_{—}000005.8 (74668855.74693681), Aug. 30, 2006, incorporated herein by reference, SEQ ID NO: 1). The 4,471 by transcript (GENBANK® Accession No. NM_{—}000859, Sep. 25, 2007, incorporated herein by reference, SEQ ID NO: 2) encodes an 888 amino acid protein (GENBANK® Accession No. NP 000850, Sep. 25, 2007, incorporated herein by reference, SEQ ID NO: 3). One of skill in the art can determine the exon/intron boundaries of an HMGCR sequence. In a particular example, HMGCR exon 1 is nucleotides 1-27 of SEQ ID NO: 1, HMGCR exon 2 is nucleotides 5310-5497 of SEQ ID NO: 1, HMGCR exon 3 is nucleotides 6580-6691 of SEQ ID NO: 1, HMGCR exon 4 is nucleotides 6972-7059 of SEQ ID NO: 1, HMGCR exon 5 is nucleotides 8301-8385 of SEQ ID NO: 1, HMGCR exon 6 is nucleotides 9931-10,036 of SEQ ID NO: 1, HMGCR exon 7 is nucleotides 12,769-12,875 of SEQ ID NO: 1, HMGCR exon 8 is nucleotides 12,985-13,101 of SEQ ID NO: 1, HMGCR exon 9 is nucleotides 13,516-13,676 of SEQ ID NO: 1, HMGCR exon 10 is nucleotides 13,795-14,042 of SEQ ID NO: 1, HMGCR exon 11 is nucleotides 14,151-14,329 of SEQ ID NO: 1, HMGCR exon 12 is nucleotides 17,230-17,424 of SEQ ID NO: 1, HMGCR exon 13 is nucleotides 17,783-17,941 of SEQ ID NO: 1, HMGCR exon 14 is nucleotides 18,092-18,249 of SEQ ID NO: 1, HMGCR exon 15 is nucleotides 19,070-19,175 of SEQ ID NO: 1, HMGCR exon 16 is nucleotides 21,384-21,554 of SEQ ID NO: 1, HMGCR exon 17 is nucleotides 21,897-22,037 of SEQ ID NO: 1, HMGCR exon 18 is nucleotides 22,125-22,283 of SEQ ID NO: 1, HMGCR exon 19 is nucleotides 22,712-22,866 of SEQ ID NO: 1, and HMGCR exon 20 is nucleotides 23,015-24,827 of SEQ ID NO: 1.

One skilled in the art will appreciate that HMGCR nucleic acid and protein molecules can vary from those publicly available, such as HMGCR sequences having one or more substitutions, deletions, insertions, or combinations thereof, while still retaining HMGCR biological activity. In one example, a variant HMGCR isoform is an HMGCR transcript (such as HMGCRv1), which lacks exon 13 as a result of alternative splicing. GenBank Accession numbers NM_{—}001130996.1 and NP001124468.1 disclose exemplary human HMGCRv1 cDNA and protein sequences, respectively (Oct. 22, 2008, incorporated herein by reference).

The HMGCR protein includes a membrane-anchor domain (exons 2-10), a flexible linker region (exons 10-11), and a catalytic domain (exons 11-20). The catalytic domain is further subdivided into an N domain, an L domain which contains an HMG-CoA binding region, and an S domain which binds NADPH.

An inhibitor binding domain of an HMGCR gene includes a portion of an HMGCR gene that encodes the region of the HMGCR protein that binds inhibitors, such as statins. Inhibitors of HMGCR bind to the catalytic domain of the protein (Istvan and Deisenhofer, Science 292:1160-1164, 2001). Thus, the inhibitor binding domain of the gene encoding human HMGCR includes exons 11 through 20 and the intervening intron sequences (i.e. introns 11-19) of the HMGCR gene. Particular examples of polymorphisms in the inhibitor binding domain include an A at nucleotide position 1176 of intron 11 of an HMGCR gene, a T at nucleotide 372 downstream of the termination codon of an HMGCR gene, or an A at nucleotide position 45 of intron 13 of an HMGCR gene.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to a genomic HMGCR DNA, an HMGCR encoding mRNA, an HMGCR encoding DNA, or an HMGCR encoding dsDNA.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization. In one example, an oligonucleotide is specifically hybridizable to DNA or RNA nucleic acid sequences including an allele of an HMGCR nucleic acid, wherein it will not hybridize to nucleic acid sequences containing a polymorphism. For instance, an oligonucleotide is specifically hybridizable to an HMGCR nucleic acid wherein position 224 of intron 5 is a G, wherein it will not hybridize to an HMGCR nucleic acid wherein position 224 of intron 5 is an A. In another example, an oligonucleotide is specifically hybridizable to an HMGCR nucleic acid wherein position 1176 of intron 11 is an A, wherein it will not hybridize to an HMGCR nucleic acid wherein position 1176 of intron 11 is a T. In a further example, an oligonucleotide is specifically hybridizable to an HMGCR nucleic acid wherein position 372 downstream of the termination codon is a T, wherein it will not hybridize to an HMGCR nucleic acid wherein position 372 downstream of the termination codon is a C. In another example, an oligonucleotide is specifically hybridizable to an HMGCR nucleic acid wherein position 45 of intron 13 is an A, wherein it will not hybridize to an HMGCR nucleic acid wherein position 45 of intron 13 is a G.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 50% Identity)

| | |
|---|---|
| Hybridization: | 6x SSC at RT to 55° C. for 16-20 hours |
| Wash at least twice: | 2x-3x SSC at RT to 55° C. for 20-30 minutes each. |

Inflammation: A localized protective response elicited by injury to tissue that serves to sequester the inflammatory agent. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is characterized by an accumulation of white blood cells, either systemically or locally at the site of inflammation. The inflammatory response may be measured by many methods well known in the art, such as the number of white blood cells, the number of polymorphonuclear neutrophils (PMN), a measure of the degree of PMN activation, such as luminol enhanced-chemiluminescence, or a measure of the amount of cytokines present. C-reactive protein is a marker of a systemic inflammatory response. A primary inflammation disorder is a disorder that is caused by the inflammation itself. A secondary inflammation disorder is inflammation that is the result of another disorder. Inflammation can lead to inflammatory diseases, such as rheumatoid arthritis, osteoarthritis, inflammatory lung disease (including chronic obstructive pulmonary lung disease), inflammatory bowl disease (including ulcerative colitis and Crohn's Disease), periodontal disease, polymyalgia rheumatica, atherosclerosis, systemic lupus erythematosus, systemic sclerosis, Sjogren's Syndrome, asthma, allergic rhinitis, and skin disorders (including dermatomyositis and psoriasis) and the like.

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Obesity: A condition in which there is an increased accumulation of body fat in humans or other mammals. Body mass index is one widely used measure of estimating body fat, calculated by dividing the subject's weight in kilograms by the subject's height in meters, squared. A body mass index of 25.0-29.9 is generally considered overweight, and greater than 30.0 is considered obese. Body fat percentage may be measured by underwater weighing, the skinfold test, or bioelectrical impedance analysis. Generally obesity is defined in men as greater than 25% body fat and in women as greater than 30% body fat.

Obesity is associated with numerous diseases, including, but not limited to, hypertension, hyperlipidemia, cardiovascular disease, diabetes mellitus type II, osteoarthritis, cancer (such as colorectal, prostate, or breast cancer), non-alcoholic fatty liver disease, obstructive sleep apnea, and asthma.

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 70 bases, for example about 10-25 bases, such as 12, 15 or 20 bases.

Polymorphism: A variation in a gene sequence, such as a variation in an HMGCR sequence. The polymorphisms can be those variations (DNA sequence differences) which are generally found between individuals or different ethnic groups and geographic locations which, while having a different sequence, produce functionally equivalent gene products. The term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Further, the term is also used interchangeably with allele as appropriate.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation. The locations of polymorphisms can be determined from an HMGCR sequence (see for example GENBANK® Accession No. NC_{—}000005.8 (74668855.74693681), Aug. 30, 2006).

For example, a polymorphism at nucleotide position 224 of intron 5 of an HMGCR gene refers to a polymorphism at the nucleotide in intron 5 that is 224 bases downstream of the G of the GT splice donor site. In another example, a polymorphism at nucleotide position 1176 of intron 11 of an HMGCR gene refers to a polymorphism at the nucleotide in intron 11 that is 1176 bases downstream of the G of the GT splice donor site. In another example, a polymorphism at nucleotide 372 downstream of the termination codon of an HMGCR gene refers to polymorphism at the nucleotide that is 372 bases downstream of the termination codon in the HMGCR 3′ untranslated region (3′ UTR). In a further example, a polymorphism at nucleotide position 45 of intron 13 of an HMGCR gene refers to a polymorphism at the nucleotide in intron 13 that is 45 bases downstream of the G of the GT splice donor site. A “downstream” nucleotide is a nucleotide 3′ to a reference point on a nucleic acid sequence. Analogous positions in other HMGCR genes can be determined by one of skill in the art using known HMGCR sequences, such as those found in GENBANK® (for example, NT_{—}006713.14 (25227457.25252283), Aug. 29, 2006; AC_{—}000048.1 (70526275.70551101), Aug. 30, 2006; or NW_{—}92279.1 (3926119.3950945), Aug. 29, 2006).

Probes and primers: A probe comprises an isolated nucleic acid capable of hybridizing to a target nucleic acid (such as an HMGCR nucleic acid molecule). A detectable label or reporter molecule can be attached to a probe or primer. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes disclosed herein are generally at least 15 nucleotides in length, such as at least 15, at least 16, at least 17, at least 18, at least 19, least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, or more contiguous nucleotides complementary to the target nucleic acid molecule, such as 20-70 nucleotides, 20-60 nucleotides, 20-50 nucleotides, 20-40 nucleotides, or 20-30 nucleotides.

Primers are short nucleic acid molecules, for instance DNA oligonucleotides 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule (such as a portion of an HMGCR nucleic acid molecule).

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers disclosed herein can be selected that include at least 15, 20, 25, 30, 35, 40, 45, 50, 55, 60, 65, 70 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure (for example, to amplify a region of an HMGCR nucleic acid molecule) include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, or more contiguous nucleotides complementary to the target nucleic acid molecule to be amplified, such as a primer of 15-70 nucleotides, 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and primers can be readily prepared based on the nucleic acid molecules provided herein. It is also appropriate to generate probes and primers based on fragments or portions of these disclosed nucleic acid molecules, for instance regions that encompass the identified polymorphisms at nucleotide position 224 of intron 5, position 1176 of intron 11, position 372 downstream of the termination codon in an HMGCR sequence, position 45 of intron 13, or the site of polymorphism in the genomic nucleic acid sequence of HMGCR or a subsequence thereof.

PCR primer pairs can be derived from a known sequence (such as a gene encoding an HMGCR protein, such as an HMGCR protein as set forth in SEQ ID NO: 3), by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Sample: A sample, such as a biological sample, is a sample obtained from a plant or animal subject. As used herein, biological samples include all clinical samples useful for detection of HMGCR in subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In a particular example, a sample includes blood obtained from a human subject, such as whole blood or serum. In another particular example, a sample includes buccal cells, for example collected using a swab or by an oral rinse.

Statin: A class of compounds which are inhibitors of 3-hydroxy-3-methylglutaryl coenzyme A reductase. Statins are hypolipidemic agents used to lower cholesterol levels. They have been shown to reduce morbidity and mortality in coronary artery disease and to reduce cerebrovascular events, particularly following an initial coronary event. The currently known statins are competitive inhibitors of HMGCR with respect to binding of the substrate HMG coenzyme A (Istvan and Deisenhofer, Science 292:1160-1164, 2001).

Statins are the most commonly used cholesterol-lowering drugs in the United States, accounting for approximately 80% of cholesterol-lowering drugs prescribed. Six statins are currently approved for use in the United States—atorvastatin (e.g. LIPITOR®), fluvastatin (e.g., LESCOL®), lovastatin (e.g., MEVACOR®), pravastatin (e.g., PRAVACHOL®), rosuvastatin (e.g., CRESTOR®), and simvastatin (e.g., ZOCOR®). An additional statin, pitavastatin, is available in Asia, but is not approved in the United States. Statins are generally well-tolerated, although severe adverse effects such as hepatotoxicity and myotoxicity occur in some instances.

Subject: Living multi-cellular vertebrate organisms, a category that includes human and non-human mammals (such as laboratory or veterinary subjects).

Therapeutically effective amount: An amount of a therapeutic agent (such as an inhibitor of HMGCR (statin)), that alone, or together with one or more additional therapeutic agents, induces the desired response, such as decreasing the risk of developing cancer or decreasing the signs and symptoms of cancer. In one example, it is an amount of statin needed to prevent or delay the development of a tumor, such as melanoma, colorectal, breast, prostate, or lung cancer, in a subject. In another example, it is an amount of statin needed to prevent or delay the metastasis of a tumor, cause regression of an existing tumor, or treat one or more signs or symptoms associated with a tumor in a subject, such as a subject having melanoma or colorectal, breast, prostate or lung cancer. Ideally, a therapeutically effective amount provides a therapeutic effect without causing a substantial cytotoxic effect in the subject. The preparations disclosed herein are administered in therapeutically effective amounts.

In one example, a desired response is to prevent the development of a tumor. In another example, a desired response is to delay the development, progression, or metastasis of a tumor, for example, by at least about 3 months, at least about six months, at least about one year, at least about two years, at least about five years, or at least about ten years. In a further example, a desired response is to decrease the occurrence of cancer, such as colorectal cancer, melanoma, breast cancer, prostate cancer, or lung cancer. In another example, a desired response is to decrease the signs and symptoms of cancer, such as the size, volume, or number of tumors or metastases. For example, the composition that includes a statin can in some examples decrease the size, volume, or number of tumors (such as colorectal tumors) by a desired amount, for example by at least 5%, at least 10%, at least 15%, at least 20%, at least 25%, at least 30%, at least 50%, at least 75%, or even at least 90%, as compared to a response in the absence of the therapeutic composition.

In general, an effective amount of a composition that includes a statin administered to a human subject will vary depending upon a number of factors associated with that subject, for example the overall health of the subject, the condition to be treated, or the severity of the condition. An effective amount of a composition that includes a statin can be determined by varying the dosage of the product and measuring the resulting therapeutic response, such as the decrease in occurrence of cancer, such as colorectal cancer, or the decrease in the size, volume or number of tumors. Statins can be administered in a single dose, or in several doses, as needed to obtain the desired response. However, the effective amount can be dependent on the source applied, the subject being treated, the severity and type of the condition being treated, and the manner of administration.

In particular examples, a therapeutically effective dose of a statin includes at least about 1 mg to about 100 mg of a statin daily. In a further example, a therapeutically effective dose of a statin includes daily statin use for at least about three months, such as at least about three months, about six months, about one year, about two years, about three years, about four years, or about five years. The disclosed compositions that include a statin can be administered alone, in the presence of a pharmaceutically acceptable carrier, in the presence of other therapeutic agents (for example other hypolipidemic agents or anti-inflammatory agents), or both.

Treatment: A therapeutic intervention. In one example, treatment refers to a therapeutic intervention that prevents or ameliorates a sign or symptom of a disease or a pathological condition related to a disease. Reducing a sign or symptom associated with a disease (such as cancer, cardiovascular disease, diabetes, obesity, inflammatory disease or auto-immune disorder) can be evidenced, for example, by a delayed onset of clinical symptoms of the disease, a reduction in severity of some or all clinical symptoms of the disease, a slower progression of the disease (for example by prolonging the life of a subject having a disease), a reduction in the number of relapses of the disease, an improvement in the overall health or well-being of the subject, or by other parameters well known in the art that are specific to the particular disease. In another example, treatment refers to a therapeutic intervention that reduces an individual's risk for developing a disease or pathological condition, such as cancer, cardiovascular disease, diabetes, obesity, inflammatory disease, or auto-immune disease.

Tumor or cancer: The product of neoplasia is a neoplasm (a tumor or cancer), which is an abnormal growth of tissue that results from excessive cell division. A tumor that does not metastasize is referred to as “benign.” A tumor that invades the surrounding tissue and/or can metastasize is referred to as “malignant.” Neoplasia is one example of a proliferative disorder.

Examples of hematological cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, and myelodysplasia.

Examples of solid cancers, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Specific non-limiting examples of cancers are colorectal, breast, prostate, and lung cancers, and melanoma.

Untranslated Region (UTR): Portion of a messenger RNA (mRNA) that is not translated, or non-coding. In eukaryotes, the non-coding portions of an mRNA include the cap, the 5′ UTR, the 3′UTR, and a poly-A tail. The 5′UTR is the non-coding portion of an mRNA that precedes the translation start codon. The 3′ UTR is the non-coding portion of an mRNA that follows the translation termination codon and extends to the start of the poly-A tail.

### Methods of Identifying Candidates for Statin Treatment

Methods are disclosed herein for identifying a subject as a candidate which is likely to benefit from treatment with an inhibitor of HMGCR. The method includes determining the presence of at least one polymorphism in the HMGCR gene in a sample from a subject. The presence of at least one polymorphism indicates that the subject is a candidate for treatment with an inhibitor of HMGCR compared to a subject which does not have at least one polymorphism. In some examples, the method includes determining the presence of at least one polymorphism in the inhibitor binding domain of the HMGCR gene in a sample from a subject.

In one example, the method includes determining if treatment of a subject with an inhibitor of HMGCR (such as a statin) will decrease their risk of developing cancer. The method includes determining the presence of at least one polymorphism in an HMGCR gene. The presence of a polymorphism indicates the subject is a candidate for treatment with a statin to decrease risk of developing cancer. The absence of a polymorphism indicates that the subject is not a candidate for treatment with a statin to decrease risk of developing cancer.

The subject can be any mammalian subject, including, but not limited to, mammals such as a dog, cat, rabbit, cow, rat, horse, pig, or monkey. In one example, the subject is a human subject. In one example, the subject is Israeli. In another example, the subject is an Ashkenazi Jew.

In some examples, the method identifies the inhibitor of HMGCR as being of use to treat cancer in the subject, such as to decrease the signs and symptoms of the cancer, to decrease the risk of developing a primary tumor, to decrease the number of metastasis, to prevent further metastasis, or to decrease the risk of developing metastatic cancer.

Methods are provided herein to determine if treatment of a subject with an inhibitor of 3-hydroxy-3-methylglutaryl coenzyme A reductase (statin) will be of use to treat cancer or decrease their risk of developing cancer. The cancer can be any cancer, including, but not limited to hematological cancer, such as leukemia, including an acute leukemia, a chronic leukemia, multiple myeloma, Waldenstrom's macroglobulinemia, heavy chain disease, myelodysplastic syndrome, or myelodysplasia. The cancer can also be a solid cancer, such as sarcoma, carcinoma, or CNS tumors.

In some examples, the cancer is colorectal cancer. In an additional example, the cancer is a melanoma. In further examples, the cancer is breast, prostate, or lung cancer.

The HMGCR inhibitor can be a statin, such as simvastatin, pravastatin, rosuvastatin, or atorvastatin. In further examples, the method identifies the subject for treatment with an inhibitor of HMGCR for at least three months, such as about three months, about six months, about one year, about two years, about three years, about four years, or about five years. In a particular embodiment, the treatment with the HMGCR inhibitor is for at least five years.

In a further embodiment, a method of identifying a candidate for treatment with an HMGCR inhibitor to decrease cancer risk further includes determining a level of CRP in a sample from the subject. In one embodiment, a subject that has an increased level of CRP as compared to a control subject is a candidate for treatment with an inhibitor of HMGCR to decrease cancer risk. In another embodiment, a subject with a CRP level that is greater than a pre-determined value, such as about 1 mg/L to about 3 mg/L, about 1 mg/L to about 2 mg/L, or about 1 mg/L, about 1.5 mg/L, about 2 mg/L, about 2.5 mg/L, or about 3 mg/L is a candidate for treatment with an inhibitor of HMGCR to reduce cancer risk.

Methods of measuring levels of CRP in a subject are known to one of skill in the art. For example, CRP levels may be measured in blood or other body fluids using an immunoassay method, such as a radioimmunoassay or enzyme-linked immunosorbent assay (see e.g. U.S. Pat. Nos. 5,272,258, 6,406,862, and 6,838,250). High sensitivity CRP assays may also be used (see Roberts et al. Clin. Chem. 47:418-425, 2001). CRP may also be measured using nephelometric immunoassay (Yamamoto et al. Vet. Quarterly 16, 74-77, 1994; Yamamoto et al., Vet. Immunol. Immunopathol. 36, 257-264, 1993) or latex agglutination test (Sarikaputi et al., Jap. J. Vet. Res. 40, 1-12, 1992; Yamada et al., Ann. Clin. Biochem. 30, 72-76, 1993).

Methods are provided herein to determine if treatment of a subject with an inhibitor of 3-hydroxy-3-methylglutaryl coenzyme A reductase will decrease their risk of developing cardiovascular disease, diabetes, obesity, inflammatory disease, or auto-immune disorder. The method includes determining the presence of at least one polymorphism in an HMGCR gene. The presence of at least one polymorphism indicates that the subject is a candidate for treatment with a statin to decrease risk of developing cardiovascular disease, diabetes, obesity, inflammatory disease, or auto-immune disorder. The absence of a polymorphism indicates that the subject is not a candidate for treatment with a statin to decrease risk of developing cardiovascular disease, diabetes, obesity, inflammatory disease, or auto-immune disorder. In some examples, the method includes determining the presence of at least one polymorphism in the inhibitor binding domain of the HMGCR gene in a sample from a subject.

In some examples, the method identifies the inhibitor of HMGCR as being of use to treat cardiovascular disease, diabetes, obesity, inflammatory disease, or auto-immune disorders in the subject, such as to decrease the signs and symptoms of cardiovascular disease, diabetes, obesity, inflammatory disease, or auto-immune disorders. The method includes determining the presence of at least one polymorphism in an HMGCR gene. The presence of at least one polymorphism indicates that the subject is a candidate for treatment with a statin to treat cardiovascular disease, diabetes, obesity, inflammatory disease, or auto-immune disorders. The absence of a polymorphism indicates that the subject is not a candidate for treatment with a statin to treat cardiovascular disease, diabetes, obesity, inflammatory disease, or auto-immune disorders. In some examples, the method includes determining the presence of at least one polymorphism in the inhibitor binding domain of the HMGCR gene in a sample from a subject.

### Exemplary Specific Polymorphisms and Methods of Detection

The methods disclosed herein include detecting the presence of a polymorphism in at least one allele of an HMGCR gene. In one embodiment, the polymorphism is at position 224 of intron 5 of an HMGCR gene, wherein the nucleotide is a G. In another embodiment, the polymorphism is at position 1176 of intron 11 of an HMGCR gene, wherein the nucleotide is an A. In a further embodiment, the polymorphism is at position 372 downstream of the termination codon of an HMGCR gene, wherein the nucleotide is a T. In another embodiment, the polymorphism is at position 45 of intron 13 of an HMGCR gene, wherein the nucleotide is an A. In additional embodiments, both alleles of an HMGCR gene are G at nucleotide position 224 of intron 5, both alleles of an HMGCR gene are A at nucleotide position 1176 of intron 11, both alleles of an HMGCR gene are T at nucleotide 372 downstream of the termination codon, or both alleles of an HMGCR gene are A at position 45 of intron 13. In further embodiments, the methods include detecting combinations of two or more of (1) the polymorphism is at position 224 of intron 5 of an HMGCR gene, wherein the nucleotide is a G; (2) the polymorphism is at position 1176 of intron 11 of an HMGCR gene, wherein the nucleotide is an A; (3) the polymorphism is at position 372 downstream of the termination codon of an HMGCR gene, wherein the nucleotide is a T; and (4) the polymorphism is at position 45 of intron 13 of an HMGCR gene, wherein the nucleotide is an A. These mutations can be detected in one or both alleles in the subject. In some examples, the method includes detecting the presence of an A nucleotide at position 1176 of intron 11 of an HMGCR gene and the presence of an A nucleotide at position 45 of intron 13 of an HMGCR gene. In one embodiment, the method includes detecting the presence of a polymorphism in the inhibitor binding domain of a gene encoding HMGCR.

Isolated nucleic acid molecules that comprise specified lengths of an HMGCR sequence and/or flanking regions can be utilized in the methods disclosed herein. Such molecules can include at least 10, 15, 20, 23, 25, 30, 35, 40, 45, 50, 55, 60, 65, or 70 consecutive nucleotides of these sequences or more, and may be obtained from any region of the disclosed sequences. By way of example, the human HMGCR and gene sequences can be apportioned into about halves or quarters based on sequence length, and the isolated nucleic acid molecules (such as oligonucleotides) can be derived from the first or second halves of the molecules, or any of the four quarters. Similarly, the human HMGCR genomic sequence can be divided into introns and exons, and HMGCR nucleic acid sequences from these introns, exons, or sequences bridging the intron/exon boundary can be used in the methods disclosed herein.

In particular embodiments, isolated nucleic acid molecules comprise or overlap at least one residue position designated as being a polymorphism that is associated with benefit from treatment with inhibitors of HMGCR. Such polymorphism sites include nucleotide position 224 of intron 5 of an HMGCR gene, such as the site of polymorphism shown by an N in SEQ ID NO: 4; position 1176 of intron 11 of an HMGCR gene, such as the site of polymorphism shown by an N in SEQ ID NO: 5; position 372 downstream of the termination codon of an HMGCR gene, such as the site of polymorphism shown by an N in SEQ ID NO: 6; and position 45 of intron 13 of an HMGCR gene, such as the site of polymorphism shown by an N in SEQ ID NO: 7.

In some embodiments, the method includes detecting the presence of at least one polymorphism in an HMGCR gene. For example, the method includes detecting the presence of a gene encoding an HMGCR protein (such as a gene encoding SEQ ID NO: 3), wherein nucleotide position 224 of intron 5, nucleotide position 1176 of intron 11, nucleotide position 372 downstream of the termination codon, and/or nucleotide position 45 of intron 13 comprises a polymorphism. In one embodiment, detection of nucleotides of a gene encoding HMGCR or sub-sequence thereof, wherein nucleotide 224 of intron 5 is a G, in a sample from a subject of interest, indicates that the subject is a candidate for treatment with a statin to decrease cancer risk. In another embodiment, detection of nucleotides of a gene encoding HMGCR, or sub-sequence thereof, wherein nucleotide 1176 of intron 11 is an A in a sample from a subject of interest indicates that the subject is a candidate for treatment with a statin to decrease cancer risk. In another embodiment, detection of nucleotides of a gene encoding HMGCR or sub-sequence thereof, wherein nucleotide 372 downstream of the termination codon, in a sample from the subject, indicates that the subject is a candidate for treatment with a statin to decrease cancer risk. In another embodiment, detection of nucleotides of a gene encoding HMGCR, or sub-sequence thereof, wherein nucleotide 45 of intron 13 is an A in a sample from a subject of interest indicates that the subject is a candidate for treatment with a statin to decrease cancer risk. Combinations of these can also be detected to indicate the subject is a candidate for treatment with a statin. In a particular embodiment, detection of nucleotides of a gene encoding HMGCR, or sub-sequence thereof, wherein nucleotide 1176 of intron 11 is an A and nucleotide 45 of intron 13 is an A in a sample from a subject of interest indicates that the subject is a candidate for treatment with a statin to decrease cancer risk.

The inhibitor binding domain of human HMGCR is encoded by about nucleotides 1240-2717 of SEQ ID NO: 2, corresponding to about amino acids 396-888 of SEQ ID NO: 3. The inhibitor binding domain of HMGCR is encompassed by about exons 11 to 20 of the gene encoding HMGCR. In one embodiment, detecting a polymorphism in the inhibitor binding domain includes detection of nucleotides of a gene encoding HMGCR, or sub-sequence thereof, wherein nucleotide 1176 of intron 11 is an A in a sample from a subject of interest indicates that the subject is a candidate for treatment with a statin to decrease cancer risk. In another embodiment, detecting a polymorphism in the inhibitor binding domain includes detection of nucleotides of a gene encoding HMGCR, or sub-sequence thereof, wherein nucleotide 372 downstream of the termination codon is a T, in a sample from the subject, indicates that the subject is a candidate for treatment with a statin to decrease cancer risk. In a further embodiment, detecting a polymorphism in the inhibitor binding domain includes detection of nucleotides of a gene encoding HMGCR, or sub-sequence thereof, wherein nucleotide 45 of intron 13 is an A, in a sample from the subject, indicates that the subject is a candidate for treatment with a statin to decrease cancer risk. Combinations of these polymorphisms can also be detected to indicate the subject is a candidate for treatment with a statin.

In some embodiments, the method includes detecting the presence of a HMGCR nucleic acid, wherein the nucleic acid sequence is the genomic sequence for human HMGCR, such as set forth in GENBANK® Accession No. NC_{—}000005.8, Aug. 30, 2006, which is incorporated herein by reference in its entirety. In one embodiment, a portion of the genomic sequence including the site of polymorphism (in bold text) is reproduced below as SEQ ID NO: 4, where N is G:

(SEQ ID NO: 4, wherein N is G or A)
CTGTATCTAACAACTCAATTATGATTCTGTAGCTACTGGAATTTG
GAATTNCCCCCATTTTTCTTTTTGAAAGTTTTCAGAACTTATGGT
AATAATAATTT.

SEQ ID NO: 4 includes the site of polymorphism and 50 bases to either side of the polymorphism in the HMGCR gene. In one embodiment, detection of SEQ ID NO: 4 or sub sequence thereof, wherein N is a G, in a sample from a subject of interest, indicates that the subject is a candidate for treatment with a statin to decrease cancer risk.

In a further embodiment, a portion of the genomic sequence including the site of polymorphism (in bold text) is reproduced below as SEQ ID NO: 5, where N is A:

(SEQ ID NO: 5, wherein N is A or T)
CCTACCTCAATTCCGCCAAACAGAAGTAACCTTTCTTTTCTGAAG
CATCCNTTATATAGAGACTGTGCATTTTTAATGGCAGTCGTACCT
TGTTGCTTATA.

SEQ ID NO: 5 includes the site of polymorphism and 50 bases to either side of the polymorphism in the HMGCR gene. In one embodiment, detection of SEQ ID NO: 5 or sub sequence thereof, wherein N is A, in a sample from a subject of interest, indicates that the subject is a candidate for treatment with a statin to decrease cancer risk.

In another embodiment, a portion of the genomic sequence including the site of polymorphism (in bold text) is reproduced below as SEQ ID NO: 6, where N is T:

(SEQ ID NO: 6, wherein N is T or C)
TGAAATTCTTGAAGTTCATGGTGATCAGTGCAATTGACCTTCTCC
CTCACNCCTGCCAGTTGAAAATGGATTTTTAAATTATACTGTAGC
TGATGAAACTC.

SEQ ID NO: 6 includes the site of polymorphism and 50 bases to either side of the polymorphism in the HMGCR gene. In one embodiment, detection of SEQ ID NO: 6 or sub sequence thereof, wherein N is T, in a sample from a subject of interest, indicates that the subject is a candidate for treatment with a statin to decrease cancer risk.

In a further embodiment, a portion of the genomic sequence including the site of polymorphism (in bold text) is reproduced below as SEQ ID NO: 7, where N is A:

(SEQ ID NO: 7, wherein N is A or T)
ATAGGTGTAAGTTGGCATTTATATATTTGCCAGTTTAAAAATACA
TCATANGTAAGGCAATGAGAAGAGTTTTAAGGACAATTAGTGAT
ACCTTTTGGGTC.

SEQ ID NO: 7 includes the site of polymorphism and 50 bases to either side of the polymorphism in the HMGCR gene. In one embodiment, detection of SEQ ID NO: 7 or sub sequence thereof, wherein N is A, in a sample from a subject of interest, indicates that the subject is a candidate for treatment with a statin to decrease cancer risk.

The biological sample may be any, which is conveniently taken from the patient and contains sufficient information to yield reliable results. Typically, the biological sample will be a biological fluid or a tissue sample that contains, for example about 1 to about 10,000,000 cells. In one embodiment, the sample contains about 1000 to about 10,000,000 cells, or from about 1,000,000 to 10,000,000 somatic cells. It is possible to obtain samples which contain smaller numbers of cells (for example about 1 to about 1,000 cells) and then enrich the cells. In addition, with certain highly sensitive assays (such as reverse transcriptase polymerase chain reaction (RT-PCR)) or by use of whole genome amplification, it is possible for the sample size to be reduced down to single cell level. The sample need not contain any intact cells, so long as it contains sufficient biological material (for example a nucleic acid, such as DNA or RNA) to assess the presence or absence of a polymorphism in nucleic acid molecules obtained from the subject.

The biological or tissue sample can be drawn from the tissue which is susceptible to the type of disease to which the detection test is directed. For example, the tissue may be obtained by surgery, biopsy, swab, or other collection method from the tissue of interest. In addition, a blood sample, serum, skin scrape, buccal cell, urine, or a sputum sample can be used. In one embodiment, the biological sample is a blood or serum sample. The blood sample may be obtained in any conventional way, such as finger prick or phlebotomy. Suitably, the blood sample is approximately 0.1 to 20 ml, or from about 1 to 15 ml, or about 10 ml of blood. In another example the sample is a buccal cell sample obtained in any conventional way, such as by a cheek swab or an oral rinse.

In another example, the sample can be previously isolated DNA. In one embodiment, the DNA is amplified by whole genome amplification (WGA) to increase the amount of DNA available for genotyping. Methods of WGA include PCR-based methods, such as ligation-mediated PCR (Saunders et al., Nucl. Acid Res., 17:9027-9037, 1989), degenerate oligonucleotide primed PCR (Telenius et al. Genomics 13:718-725, 1992; U.S. Pat. No. 5,731,171), and primer extension preamplification PCR (Zhang et al., Proc. Natl. Acad. Sci., 89:5847-5851, 1992; U.S. Pat. No. 6,365,375), and non-PCR-based methods, such as multiple displacement amplification (U.S. Pat. Nos. 6,124,120 and 6,977,148; Dean et al., Proc. Natl. Acad. Sci., 99:5261-5266, 2002).

Southern hybridization is also an effective method of identifying differences in sequences. Hybridization conditions, such as salt concentration and temperature can be adjusted for the sequence to be screened. Southern blotting and hybridization protocols are described in Current Protocols in Molecular Biology (Greene Publishing Associates and Wiley-Interscience, pages 2.9.1-2.9.10). Very high specific activity probe can be obtained using commercially available kits such as the Ready-To-Go DNA Labeling Beads (Pharmacia Biotech), following the manufacturer's protocol.

Restriction enzyme polymorphism is an additional method of identifying differences in sequences. Restriction enzyme polymorphism allows differences to be established by comparing the characteristic polymorphic patterns that are obtained when certain regions of genomic DNA are cut with various restriction enzymes. In one embodiment, the genomic DNA is amplified prior to being cut with the restriction enzymes.

In one embodiment, an HMGCR nucleic acid that comprises the inhibitor binding domain, or a portion thereof is amplified. In another embodiment, a gene encoding HMGCR, or a portion thereof (such as an intron or untranslated region, for example, intron 5, 11, 13, the 3′UTR, or a portion thereof) is amplified. Amplification of a selected, or target, nucleic acid sequence from a gene encoding HMGCR can be carried out by any suitable means (see for example Kwoh and Kwoh, Am Biotechnol Lab, 8, 14, 1990). Examples of suitable amplification techniques include, but are not limited to, polymerase chain reaction, ligase chain reaction (see for example Barany, Proc Natl Acad Sci USA 88:189, 1991), strand displacement amplification (see for example Walker et al., Nucleic Acids Res. 20:1691, 1992; Walker et al., Proc Natl Acad Sci USA 89:392, 1992), transcription-based amplification (see for example Kwoh et al., Proc Natl Acad Sci USA, 86:1173, 1989), self-sustained sequence replication (or “3SR”) (see for example Guatelli et al., Proc Natl Acad Sci USA, 87:1874, 1990), the Q β-replicase system (see for example Lizardi et al., Biotechnology, 6:1197, 1988), nucleic acid sequence-based amplification (or “NASBA”) (see for example Lewis, Genetic Engineering News, 12(9):1, 1992), the repair chain reaction (or “RCR”) (see for example Lewis, Genetic Engineering News, 12(9):1, 1992), and boomerang DNA amplification (or “BDA”) (see for example Lewis, Genetic Engineering News, 12(9):1, 1992). In one specific non-limiting example, polymerase chain reaction is utilized.

Single strand polymorphism assay (“SSPA”) analysis and the closely related heteroduplex analysis methods can be used as effective methods for screening for single-base polymorphisms (Orita, et al., Proc Natl Acad Sci USA, 86:2766, 1989). In these methods, the mobility of PCR-amplified test DNA from clinical specimens is compared with the mobility of DNA amplified from normal sources by direct electrophoresis of samples in adjacent lanes of native polyacrylamide or other types of matrix gels. Single-base changes often alter the secondary structure of the molecule sufficiently to cause slight mobility differences between the normal and mutant PCR products after prolonged electrophoresis.

Ligase chain reaction is yet another recently developed method of screening for mutated nucleic acids. Ligase chain reaction (LCR) is also carried out in accordance with known techniques. LCR is especially useful to amplify, and thereby detect, single nucleotide differences between two DNA samples. In general, the reaction is carried out with two pairs of oligonucleotide probes: one pair binds to one strand of the sequence to be detected; the other pair binds to the other strand of the sequence to be detected. The reaction is carried out by, first, denaturing (e.g., separating) the strands of the sequence to be detected, then reacting the strands with the two pairs of oligonucleotide probes in the presence of a heat stable ligase so that each pair of oligonucleotide probes hybridize to target DNA and, if there is perfect complementarity at their junction, adjacent probes are ligated together. The hybridized molecules are then separated under denaturation conditions. The process is cyclically repeated until the sequence has been amplified to the desired degree. Detection may then be carried out in a manner like that described above with respect to PCR.

For amplification of mRNAs, it is within the scope of the present disclosure to reverse transcribe mRNA into cDNA followed by polymerase chain reaction (RT-PCR); or, to use a single enzyme for both steps as described in U.S. Pat. No. 5,322,770 or, to use Asymmetric Gap LCR (RT-AGLCR) as described by Marshall et al. PCR Methods Appl. 4:80-84, 1994. AGLCR is a modification of GLCR that allows the amplification of RNA.

A variety of PCR techniques are familiar to those skilled in the art. For a review of PCR technology, see White (PCR Cloning Protocols, Methods in Molecular Biology, Vol. 67, 1997) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or VENT® polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites (see also U.S. Pat. Nos. 4,683,195, 4,683,202 and U.S. Pat. No. 4,965,188).

In one embodiment, DNA amplification techniques such as the foregoing involve the use of a probe, a pair of probes, or two pairs of probes which specifically bind to nucleic acid sequences including one allele of HMGCR (such as a G at nucleotide 224 of intron 5), but do not bind to nucleic acid sequences containing a polymorphism (such as an A at nucleotide 224 of intron 5), under the same hybridization conditions, and which serve as the primer or primers for the amplification reaction. In another embodiment, the method involves the use of a probe, a pair of probes, or two pairs of probes which specifically bind to nucleic acid sequences including one allele of HMGCR (such as an A at nucleotide 1176 of intron 11), but do not bind to nucleic acid sequences containing a polymorphism (such as a T at nucleotide 1176 of intron 11), under the same hybridization conditions, and which serve as the primer or primers for the amplification reaction. In a further embodiment, the method involves the use of a probe, a pair of probes, or two pairs of probes which specifically bind to nucleic acid sequences including one allele of HMGCR (such as a T at nucleotide 372 downstream of the termination codon), but do not bind to nucleic acid sequences containing a polymorphism (such as a C at nucleotide 372 downstream of the termination codon), under the same hybridization conditions, and which serve as the primer or primers for the amplification reaction. In another embodiment, the method involves the use of a probe, a pair of probes, or two pairs of probes which specifically bind to nucleic acid sequences including one allele of HMGCR (such as an A at nucleotide 45 of intron 13), but do not bind to nucleic acid sequences containing a polymorphism (such as a G at nucleotide 45 of intron 13), under the same hybridization conditions, and which serve as the primer or primers for the amplification reaction. In additional embodiments, the probes may be used in combination in order to detect the presence of more than one polymorphism in a subject.

In a further embodiment, the primers can bind a nucleic acid containing both alleles of the HMGCR polymorphism. An amplification reaction is performed and the resulting nucleic acid is sequenced. Screening for mutated nucleic acids can be accomplished by direct sequencing of nucleic acids. A nucleic acid containing the polymorphic HMGCR nucleic acid can be sequenced to determine the exact nature of the polymorphism. Nucleic acid sequences can be determined through a number of different techniques which are well known to those skilled in the art. Nucleic acid sequencing can be performed by chemical or enzymatic methods. The enzymatic method relies on the ability of DNA polymerase to extend a primer, hybridized to the template to be sequenced, until a chain-terminating nucleotide is incorporated. The most common methods utilize dideoxynucleotides. Primers may be labeled with radioactive or fluorescent labels. Various DNA polymerases are available including Klenow fragment, AMV reverse transcriptase, Taq DNA polymerase, and modified T7 polymerase.

Microsequencing reactions can also be performed on a nucleic acid including a polymorphism of HMGCR contained in amplified nucleic acids from samples taken from individuals of interest. In some embodiments, DNA samples are subjected to PCR amplification of an HMGCR gene, or portions thereof. The genomic amplification products are then subjected to automated microsequencing reactions using ddNTPs (specific fluorescence for each ddNTP) and appropriate oligonucleotide microsequencing primers which can hybridize just upstream of the polymorphic base of interest. Once specifically extended at the 3′ end by a DNA polymerase using a complementary fluorescent dideoxynucleotide analog (thermal cycling), the primer is precipitated to remove the unincorporated fluorescent ddNTPs. The reaction products in which fluorescent ddNTPs have been incorporated are then analyzed by electrophoresis on automated sequencing machines to determine the identity of the incorporated base, thereby identifying the polymorphic marker present in the sample.

As a further alternative to the process described above, several solid phase microsequencing reactions have been developed. The basic microsequencing protocol is the same as described previously, except that either the oligonucleotide microsequencing primers or the PCR-amplified products of the DNA fragment of interest are immobilized. For example, immobilization can be carried out by an interaction between biotinylated DNA and streptavidin-coated microtitration wells or avidin-coated polystyrene particles.

In such solid phase microsequencing reactions, incorporated ddNTPs can either be radiolabeled or linked to a fluorescent marker, such as fluorescein. The detection of radiolabeled ddNTPs can be achieved through scintillation-based techniques. The detection of fluorescein-linked ddNTPs can be based on the binding of anti-fluorescein antibody conjugated with alkaline phosphatase, followed by incubation with a chromogenic substrate (such as p-nitrophenyl phosphate).

Other possible reporter-detection couples include: ddNTP linked to dinitrophenyl (DNP) and anti-DNP alkaline phosphatase conjugate and biotinylated ddNTP and horseradish peroxidase-conjugated streptavidin with o-phenylenediamine as a substrate (see for example PCT Publication No. WO 92/15712). A diagnosis kit based on fluorescein-linked ddNTP with antifluorescein antibody conjugated with alkaline phosphatase is commercialized under the name PRONTO® by GamidaGen Ltd.

Solid-phase DNA sequencing can also be utilized that relies on the detection of DNA polymerase activity by an enzymatic luminometric inorganic pyrophosphate detection assay (ELIDA). The PCR-amplified products are biotinylated and immobilized on beads. The microsequencing primer is annealed and four aliquots of this mixture are separately incubated with DNA polymerase and one of the four different ddNTPs. After the reaction, the resulting fragments are washed and used as substrates in a primer extension reaction with all four dNTPs present. The progress of the DNA-directed polymerization reactions are monitored with the ELIDA. Incorporation of a ddNTP in the first reaction prevents the formation of pyrophosphate during the subsequent dNTP reaction. In contrast, no ddNTP incorporation in the first reaction gives extensive pyrophosphate release during the dNTP reaction and this leads to generation of light throughout the ELIDA reactions. From the ELIDA results, the first base after the primer is easily deduced. Methods for multiplex detection of single nucleotide polymorphism are also known in the art which the solid phase minisequencing principle is applied to an oligonucleotide array format.

An amplified HMGCR nucleic acid can be detected in real-time, for example by real-time PCR, in order to determine the presence, and/or the amount of a polymorphism of an HMGCR nucleic acid. In this manner, an amplified nucleic acid sequence, such as an amplified polymorphic HMGCR nucleic acid sequence, can be detected using a probe specific for the product amplified from the HMGCR nucleic acid sequence of interest, such as amplified polymorphic HMGCR nucleic acid sequences.

Real-time PCR monitors the fluorescence emitted during the reaction as an indicator of amplicon production during each PCR cycle as opposed to the endpoint detection. The real-time progress of the reaction can be viewed in some systems. Typically, real-time PCR uses the detection of a fluorescent reporter. Typically, the fluorescent reporter's signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed.

In one embodiment, the fluorescently-labeled probes rely upon fluorescence resonance energy transfer (FRET), or in a change in the fluorescence emission wavelength of a sample, as a method to detect hybridization of a DNA probe to the amplified target nucleic acid in real-time. For example, FRET that occurs between fluorogenic labels on different probes (for example, using HybProbes) or between a fluorophore and a non-fluorescent quencher on the same probe (for example, using a molecular beacon or a TAQMAN® probe) can identify a probe that specifically hybridizes to the DNA sequence of interest and in this way, using a probe for an HMGCR polymorphism, can detect the presence of the HMGCR polymorphism in a sample. In some embodiments, the fluorescently-labeled DNA probes used to identify amplification products have spectrally distinct emission wavelengths, thus allowing them to be distinguished within the same reaction tube, for example in multiplex PCR, for example a multiplex real-time PCR.

In another embodiment, a melting curve analysis of the amplified target nucleic acid can be performed subsequent to the amplification process. The Tₘ of a nucleic acid sequence depends on the length of the sequence and its G/C content. Thus, the identification of the Tₘ for a nucleic acid sequence can be used to identify the amplified nucleic acid, for example by using double-stranded DNA binding dye chemistry, which quantitates the amplicon production by the use of a non-sequence specific fluorescent intercalating agent (such as SYBR®-green or ethidium bromide). SYBR® green is a fluorogenic minor groove binding dye that exhibits little fluorescence when in solution but emits a strong fluorescent signal upon binding to double-stranded DNA. Typically, SYBR® green is used in singleplex reactions, however when coupled with melting point analysis, it can be used for multiplex reactions.

Any type of thermal cycler apparatus can be used for the amplification of HMGCR nucleic acid and/or the determination of hybridization. Examples of suitable apparatuses include a PTC-100® Peltier Thermal Cycler (MJ Research, Inc.; San Francisco, Calif.), a ROBOCYCLER® 40 Temperature Cycler (Stratagene; La Jolla, Calif.), or a GENEAMP® PCR System 9700 (Applied Biosystems; Foster City, Calif.). For real-time PCR, any type of real-time thermocycler apparatus can be used. For example, a BioRad iCycler IQ™, LIGHTCYCLER™ (Roche; Mannheim, Germany), a 7700 Sequence Detector (Perkin Elmer/Applied Biosystems; Foster City, Calif.), ABI systems such as the 7000, 7500, 7700, or 7900 systems (Applied Biosystems; Foster City, Calif.), or an MX4000™, MX3000™ or MX3005™ (Stratagene; La Jolla, Calif.); DNA Engine Opticon Continuous Fluorescence Detection System (MJ Research); and Cepheid SMARTCYCLER™ can be used to amplify nucleic acid sequences in real-time.

In one example, an allele-specific oligonucleotide extension-ligation assay utilizing microbeads, such as the Illumina GOLDENGATE® assay (see e.g. Fan et al. Cold Spring Harbor Symp. Quant. Biol. LXVIII:69-78, 2003), can be used to determine the presence of a polymorphism in a gene encoding HMGCR. Typically, two allele-specific oligonucleotides (ASO), one that is specific to each allele of a SNP are included in the assay, such as oligonucleotides that include either G or A at nucleotide position 224 of intron 5 of an HMGCR gene (for example, SEQ ID NOs: 8 and 9), oligonucleotides that include either A or T at nucleotide position 1176 of intron 11 of an HMGCR gene (for example, SEQ ID NOs: 11 and 12), oligonucleotides that include either T or C at nucleotide position 372 downstream of the termination codon of an HMGCR gene (for example, SEQ ID NOs: 14 and 15), or oligonucleotides that include either A or G at nucleotide position 45 of intron 13 of an HMGCR gene. A locus-specific oligonucleotide (LSO) that hybridizes downstream of the SNP site (for example SEQ ID NOs: 10, 13, or 16) is also included in the assay. All three oligonucleotides contain regions of genomic complementarity, such as complementarity to the gene encoding an HMGCR protein (for example the gene encoding SEQ ID NO: 3), and universal PCR primer sites. The LSO also contains a unique address sequence that targets it to a particular bead type. Oligonucleotides are hybridized to a DNA sample and extension from the ASO and ligation of the extended product to the LSO is carried out. These ligated products are used as a template for PCR using universal PCR primers. The universal primers associated with the ASOs include distinct fluorescent dyes, such as Cy3 or Cy5. The resulting dye-labeled DNAs are hybridized to their complement bead type through the address sequence included in the locus-specific oligonucleotide. The beads are contained in a microarray, chip, or plate, such as a VERACODE™ Bead plate, which is analyzed for fluorescence signal, for example using a BEADXPRESS™ Reader. Genotypes are determined using a software analysis package, such as the BeadStation data analysis module. Exemplary oligonucleotides which may be used in an allele-specific oligonucleotide extension-ligation assay to detect the presence of HMGCR polymorphisms are given in Table 2 below (SEQ ID NOs: 8-28). A multiplex reaction may be carried out to detect multiple HMGCR polymorphisms simultaneously.

In further embodiments, the method of determining the presence of a polymorphism in a gene encoding HMGCR includes determining the presence of at least one polymorphism in an RNA sample. For example, presence of an HMGCR polymorphism may be detected by reverse transcription of mRNA into cDNA followed by polymerase chain reaction (RT-PCR); or use a single enzyme for both steps as described in U.S. Pat. No. 5,322,770. Determination of a polymorphism may also be detected by Northern blot analysis, for example detecting a variant that alters the size or expression level of an HMGCR RNA. Measurement of RNA levels that may be altered by the presence of an HMGCR polymorphism may also be measured using an RNase protection assay.

### Kits

In one embodiment there are provided methods, compositions, and kits for determining the presence of an HMGCR polymorphism in an individual. The genotyping method comprises identifying the nucleotides in one or both copies of the HMGCR gene(s) from the individual.

Specific contemplated genotyping compositions comprise an oligonucleotide probe or primer that overlaps (e.g. includes) and is designed to specifically hybridize to a target region containing, or adjacent to, a nucleic acid encoding an HMGCR protein (such as a nucleic acid encoding SEQ ID NO: 3), or a portion thereof. For example, oligonucleotide probes and/or primers that are designed to identify the nucleotide at position 224 of intron 5 of an HMGCR gene can be included in the kit. In another example, oligonucleotide probes and/or primers that are designed to identify the nucleotide at position 1176 of intron 11 of an HMGCR gene can be included in the kit. In a further example, oligonucleotide probes and/or primers that are designed to identify the nucleotide at position 372 downstream of the termination codon of an HMGCR gene can be included in the kit. In yet another example, oligonucleotide probes and/or primers that are designed to identify the nucleotide at position 45 of intron 13 of an HMGCR gene can be included in the kit. In a particular embodiment, the kit may contain probes and/or primers to detect combinations of two or more of (1) the polymorphism at position 224 of intron 5 of an HMGCR gene; (2) the polymorphism at position 1176 of intron 11 of an HMGCR gene; (3) the polymorphism at position 372 downstream of the termination codon of an HMGCR gene; and (4) the polymorphism at position 45 of intron 13 of an HMGCR gene. Exemplary oligonucleotides are listed in Table 2 (SEQ ID NOs: 8-16).

A representative genotyping kit comprises one or more oligonucleotide(s) designed to genotype one HMGCR. The provided genotyping methods, compositions, and kits are useful, for instance, for identifying an individual, or collection of individuals, that has one of the genotypes described herein, and to determine if the individual is a candidate for treatment with an HMGCR inhibitor to decrease risk of developing cancer in that individual. Exemplary probes and primers for HMGCR are disclosed in the examples below; the kit can include any number of the specific oligonucleotides disclosed in the examples section. A kit can optionally include instructional material, such as directions for use in written, video or digital format.

The present disclosure is illustrated by the following non-limiting Examples.

### EXAMPLES

### Example 1

### Subjects and Samples

This example describes the demographics of the subjects analyzed and the DNA samples used in the analysis.

Subjects were individuals who completed all required elements of the Molecular Epidemiology of Colorectal Cancer (MECC) study (Poynter, et al., supra). The MECC study was a population-based case-control study of patients diagnosed with colorectal cancer between 1998 and 2004 in northern Israel and controls matched according to age, sex, clinic location, and ethnic group. The ethnic background of the study population was 66% Ashkenazi Jewish. The subjects included 1,973 cases and 2,073 population-based controls. The study population included 388 subjects taking statins (130 cases and 258 controls). No severe adverse effects occurred in study participants taking statins.

Whole genome amplification of 4,036 DNA samples from the MECC study was completed using Qiagen Phi29 whole genome amplification (WGA). WGA was successful for 97.1% of the samples, with no differences in the amplification of case and control DNA. The quality of the WGA DNA was measured by examining the success of PCR reactions over a range of chromosomal locations. The amplified DNA was classified as usable, unusable, or no amplification, based on the test PCR reactions. A total of 3,933 (97.9%) of the samples were classified as usable, 112 (2.1%) were classified as unusable, and 1 (0.01%) did not amplify. The average yield was 62.8 μg of DNA.

### Example 2

### Selection of Haplotype Tagging SNPs

This example describes the selection of haplotype tagging single nucleotide polymorphisms (htSNPs) for genes in the cholesterol synthesis pathway and gene targets affected by geranyl-geranylation (a metabolic product that branches off from the cholesterol synthesis pathway).

Using the Human Haplotype Map and the Haplotyper Bioinformatic Suite, htSNPs were selected with a minimum minor allele frequency (MAP) of greater than 0.01 and greater than or equal to 80% association with at least two additional SNPs (R²≧0.8). For the associated SNPs, the MAP was set at a threshold of 0.1 or greater. These haplotype blocks are in genes including HMGCR (3-hydroxy-3-methylglutaryl coenzyme A reductase), RABGGTA (Rab geranylgeranyltransferase alpha subunit), RABGGTB (Rab geranylgeranyltransferase beta subunit), PGGT1B (protein geranylgeranyltransferase type 1 beta subunit), FNTA (farnesyltransferase CAAX box, alpha), FDFT1 (farnesyl-diphosphate farnesyltransferase 1), CETP (cholesteryl ester transfer protein), LDLR (low-density lipoprotein receptor), APOB (apolipoprotein B), APOE (apolipoprotein E), ABCG5 (ATP-binding cassette, sub-family G (WHITE), member 5), ABCG8 (ATP-binding cassette, sub-family G (WHITE), member 8), CRP (C-reactive protein), NSDHL (NAD(P) dependent steroid dehydrogenase-like), SC4MOL (sterol-C4-methyl oxidase-like), and LIPC (hepatic triacylglycerol lipase). A total of 200 htSNPs were selected. All major haplotypes fitting these criteria were captured by the htSNPs.

### Example 3

### Intent-to-Genotype Population Genotyping

This example describes genotyping of the selected htSNPs and analysis of case and control subjects, regardless of statin usage.

Genotyping of the selected htSNPs was carried out in both colorectal cancer cases and controls. Data were analyzed without regard to statin usage (referred to as intent-to-genotype population).

Genotyping was done using Illumina GOLDENGATE® assays. DNA was quantitated using QUANT-IT™ PICOGREEN® dsDNA reagent. Activated biotinylated DNA was prepared by adding reagent MS1 (for single use plate) or reagent MM1 (for multi-use plate). 250 ng of DNA was added to each well for single use plates and 2 μg of DNA was added to each well for multi-use plates. Activated DNA was precipitated with reagent PS1 and 2-propanol, air dried, and resuspended in reagent RS1.

An allele-specific extension plate was prepared by adding the activated DNA to a plate containing reagent OB1 and the oligonucleotide pool designed to detect the selected SNPs (described in Example 2). Oligonucleotide hybridization was performed by heating the plate to 70° C. and allowing it to gradually cool to 30° C. Beads were washed two times with reagent AM1 and two times with reagent UB1 to remove non-specifically hybridized and excess oligonucleotides. Extension and ligation enzymes (reagent MEL) were added and the plate was incubated at 45° C. for 15 minutes. Samples were washed with reagent UB1 and eluted by incubating for 1 minute at 95° C. with reagent IP1. The eluted samples were transferred to a plate containing reagent MMP, uracil DNA glycosylase, and Taq DNA polymerase (ABI). PCR conditions were as follows: 10 minutes at 37° C., 3 minutes at 95° C., {35 seconds at 95° C., 35 seconds at 56° C., 2 minutes at 72° C.}×34, 10 minutes at 72° C., 5 minutes at 4° C.

Following PCR, the contents of each well were mixed with reagent MPB and transferred to a filter plate. The filter plate was centrifuged at 1000×g for 5 minutes at room temperature, then washed with reagent UB2 and re-centrifuged. 0.1 N NaOH was added to the wells of the filter plate and centrifuged, with the eluate collected in a new plate containing reagent MH1. Samples were aliquoted to a VERACODE™ Bead Plate and hybridization was carried out by incubating for 30 minutes at 60° C. followed by gradual cooling to 45° C. The plate was washed two times with reagent UB2 and one time with reagent WC1. The plate was scanned using a BEADXPRESS™ Reader and analyzed with the BeadStation Data Analysis module.

After adjusting for age, gender, and ethnicity in the intent-to-genotype population, three genes that had significant association with CRC risk were identified. Each gene had two SNPs that were significantly associated with a role in modifying CRC risk (Table 1).

TABLE 1
Effect of SNPs on CRC Risk in Intent-to-Genotype Population

| | | 95% Confidence Interval | | |
|---|---|---|---|---|
| SNP | Odds ratio | Lower | Upper | p-value |
| LDLR_rs2569538 | 1.34 | 1.10 | 1.63 | 0.00375 |
| LDLR_rs11669576 | 1.58 | 1.02 | 2.44 | 0.03704 |
| LIPC_rs16940372 | 0.79 | 0.67 | 0.94 | 0.00707 |
| LIPC_rs4774302 | 1.14 | 1.01 | 1.30 | 0.03379 |
| ABCG8_rs4299376 | 0.86 | 0.76 | 0.98 | 0.02217 |
| ABCG8_rs4245791 | 0.86 | 0.76 | 0.98 | 0.02266 |

Two independent SNP variants in the LDLR gene were associated with increased risk of CRC. LDLR encodes the low-density lipoprotein receptor. Lipoprotein receptor-related proteins have been shown to mediate Wnt signaling, most notably LRP5.

One common allele (rs16940372) and one variant allele of LIPC were associated with a moderate increase in CRC risk. LIPC encodes the hepatic triacylglycerol lipase, which is expressed in liver, colon epithelium, and many other tissues.

Two variants in ABCG8 were associated with a moderate level of CRC risk reduction. ABCG8 encodes ATP-binding cassette sub-family G member 8 and is the genetic locus for the disease sitosterolemia and the target of the cholesterol lowering drug ezetimibe (ZETIA®).

### Example 4

### Genotyping of Intent-to-Treat Arms

This example describes genotyping of the selected htSNPs, with analysis based on statin use.

Genotyping was carried out as described in Example 3. Oligonucleotides used to genotype HMGCR htSNPs are described in Table 2.

TABLE 2
HMGCR htSNP Genotyping Oligonucleotides

| HMGCR SNP | Oligonucleotide Sequence | SEQ ID NO: |
|---|---|---|
| rs2303152 | ACTTCGTCAGTAACGGACAAACT | SEQ ID NO: 8 |
| | TTCAAAAAGAAAAATGGGGGT | |
| | GAGTCGAGGTCATATCGTAAACT | SEQ ID NO: 9 |
| | TTCAAAAAGAAAAATGGGGGC | |
| | ATTCCAAATTCCAGTAGCTACAG | SEQ ID NO: 10 |
| | GAAGCCGCTCTTCTTAGTGATCT | |
| | GGTCTGCCTATAGTGAGTC | |
| rs12654264 | ACTTCGTCAGTAACGGACAGTAA | SEQ ID NO: 11 |
| | CCTTTCTTTTCTGAAGCATCCT | |
| | GAGTCGAGGTCATATCGTAGTAA | SEQ ID NO: 12 |
| | CCTTTCTTTTCTGAAGCATCCA | |
| | TATATAGAGACTGTGCATTTTTA | SEQ ID NO: 13 |
| | ATGTTCAAAGGTAGACCCGACAC | |
| | GTTTGTCTGCCTATAGTGAGTC | |
| rs12916 | ACTTCGTCAGTAACGGACGTGCA | SEQ ID NO: 14 |
| | ATTGACCTTCTCCCTCACT | |
| | GAGTCGAGGTCATATCGTGTGCA | SEQ ID NO: 15 |
| | ATTGACCTTCTCCCTCACC | |
| | CTGCCAGTTGAAAATGGATTTGA | SEQ ID NO: 16 |
| | ATTTACGCATTGTGACTGGACGT | |
| | CTGCCTATAGTGAGTC | |
| rs4704209 | ACTTCGTCAGTAACGGACCCATG | SEQ ID NO: 17 |
| | TAATTCCATAATGTGGCTATCTAT | |
| | GAGTCGAGGTCATATCGTCCATG | SEQ ID NO: 18 |
| | TAATTCCATAATGTGGCTATCTAC | |
| | GTCTAGAAACTAGACCATAAAGG | SEQ ID NO: 19 |
| | AAATCAGTCGTGCGATGTTCCAA | |
| | GCGTCTGCCTATAGTGAGTC | |
| rs2241402 | ACTTCGTCAGTAACGGACGCCAA | SEQ ID NO: 20 |
| | AATTGTAGAAAAAAAGAAATCTT | |
| | AT | |
| | GAGTCGAGGTCATATCGTGCCAA | SEQ ID NO: 21 |
| | AATTGTAGAAAAAAAGAAATCTT | |
| | AA | |
| | AATAATGAGATTGGAACTGAGGA | SEQ ID NO: 22 |
| | ATCGGGATGGTCACAACATTTCG | |
| | TGTCTGCCTATAGTGAGTC | |
| rs5908 | ACTTCGTCAGTAACGGACGTGCA | SEQ ID NO: 23 |
| | CGTCTACAGAAACTTCATACAAG | |
| | TA | |
| rs5908 | GAGTCGAGGTCATATCGTGTGCA | SEQ ID NO: 24 |
| | CGTCTACAGAAACTTCATACAAG | |
| | TG | |
| | AGCTGGACGCAACCTTTATATTC | SEQ ID NO: 25 |
| | TCCGGCTGATGGAACCGTAGGTC | |
| | TGCCTATAGTGAGTC | |
| rs10515198 | ACTTCGTCAGTAACGGACCAATT | SEQ ID NO: 26 |
| | CTTAAATCTTGTGCTATGAAGAA | |
| | AT | |
| | GAGTCGAGGTCATATCGTCAATT | SEQ ID NO: 27 |
| | CTTAAATCTTGTGCTATGAAGAA | |
| | AC | |
| | CTATTAATCCTTCCTATTAATGT | SEQ ID NO: 28 |
| | AAAGATGCCAATATGACGATTGC | |
| | TAGAGTCTGCCTATAGTGAGTC | |

The data were analyzed based on use or non-use of statins in cases and controls. This analysis identified three htSNPs in HMGCR associated with significant modification of CRC risk in the case-only analysis (Tables 3-5). No significant association was found between these three htSNPs and CRC risk in control subjects.

TABLE 3
CRC Risk Associated with HMGCR rs2303152

| | | | | 95% Confidence | |
|---|---|---|---|---|---|
| | | | | Interval | |
| | Control (n) | Case (n) | Odds Ratio | Lower | Upper |
| G/G | | | | | |
| Non-Use | 567 | 640 | 1.00 | N/A | N/A |
| Statin Use | 130 | 49 | 0.33 | 0.24 | 0.47 |
| G/A | | | | | |
| Non-Use | 243 | 254 | 1.00 | N/A | N/A |
| Statin Use | 37 | 27 | 0.7 | 0.41 | 1.18 |
| A/A | | | | | |
| Non-Use | 23 | 30 | 1.00 | N/A | N/A |
| Statin Use | 5 | 6 | 0.92 | 0.25 | 3.39 |

TABLE 4
CRC Risk Associated with HMGCR rs12654264

| | | | | 95% Confidence | |
|---|---|---|---|---|---|
| | | | | Interval | |
| | Control (n) | Case (n) | Odds Ratio | Lower | Upper |
| A/A | | | | | |
| Non-Use | 276 | 289 | 1.00 | N/A | N/A |
| Statin Use | 55 | 15 | 0.26 | 0.14 | 0.47 |
| T/A | | | | | |
| Non-Use | 410 | 452 | 1.00 | N/A | N/A |
| Statin Use | 84 | 43 | 0.46 | 0.31 | 0.69 |
| T/T | | | | | |
| Non-Use | 144 | 176 | 1.00 | N/A | N/A |
| Statin Use | 33 | 24 | 0.6 | 0.34 | 1.05 |

TABLE 5
CRC Risk Associated with HMGCR rs12916

| | | | | 95% Confidence | |
|---|---|---|---|---|---|
| | | | | Interval | |
| | Control (n) | Case (n) | Odds Ratio | Lower | Upper |
| T/T | | | | | |
| Non-Use | 72 | 46 | 1.00 | N/A | N/A |
| Statin Use | 28 | 4 | 0.22 | 0.07 | 0.68 |
| T/C | | | | | |
| Non-Use | 78 | 70 | 1.00 | N/A | N/A |
| Statin Use | 32 | 18 | 0.63 | 0.32 | 1.21 |
| C/C | | | | | |
| Non-Use | 22 | 18 | 1.00 | N/A | N/A |
| Statin Use | 14 | 8 | 0.7 | 0.24 | 2.03 |

SNP rs2303152 is located in intron 5 at a position 224 bases downstream of the G of the GT splice donor site. Two of the SNPs are localized in the portion of the HMGCR gene that includes that inhibitor binding domain. The inhibitor binding domain of the HMGCR gene includes exons 11-20 and the intervening introns (introns 11-19). SNP rs12654264 is located in intron 11 at a position 1176 bases downstream of the G of the GT splice donor site. SNP rs12916 is located in exon 20, in the 3′ UTR region of the HMGCR gene at a position 372 bases downstream of the termination codon.

For all three HMGCR SNPs identified, CRC risk was decreased in a gene dosage manner in the codominant inheritance model. For example, risk of CRC in statin users having the G/G genotype for HMGCR SNP rs2303152 was decreased compared with non-statin users, with risk of CRC significantly increasing for statin users having the G/A genotype or A/A genotype in a dose dependent manner (p=0.0098). Risk of CRC in statin users having the A/A genotype for HMGCR SNP rs12654264 was decreased compared with non-statin users, with risk of CRC significantly increasing for statin users having the A/T genotype or T/T genotype in a dose dependent manner (p=0.0445). Risk of CRC in statin users having the T/T genotype for HMGCR SNP rs12619 was decreased compared with non-statin users, with risk of CRC increasing for statin users having the T/C genotype or C/C genotype in a dose dependent manner, although the data showed a trend that did not reach significance (p=0.1905).

### Example 5

### Association of HMGCR Genotype, Statin Use and Cancer Risk

This example describes the assessment of cancer risk in subjects using statins and association with HMGCR genotype.

A population of subjects with cancer (such as skin, colorectal, stomach, lung, breast, prostate, kidney, bladder, or pancreatic cancer, or lymphoma, melanoma, or other cancer) will be matched with population-based control subjects.

Cases and controls will be genotyped for the HMGCR SNPs identified in Example 4. The data will be analyzed based on use or non-use of statins in cases and controls. The association between cancer risk and the HMGCR SNPs will be assessed in both case and control subjects.

### Example 6

### Identification of HMGCR Inhibitor Binding Domain Variants Associated with Decreased Cancer Risk in Statin Users

This example describes identification of variants in the inhibitor binding domain of HMGCR that are associated with decreased cancer risk in individuals taking statins as compared with individuals who are not taking statins.

The gene encoding HMGCR consists of twenty exons. The inhibitor binding domain includes exons 11-20 (the catalytic domain). PCR primer sequences are designed to amplify exons 11-20 of the HMGCR gene. Primer sequences are designed from flanking intronic sequences to allow the assessment of the sequence of the inhibitor binding domain coding sequence and intron-exon splice junctions of the HMGCR gene in genomic DNA from case and control subjects.

Exons 11-20 and adjacent splice sites of the HMGCR gene are amplified from genomic DNA. PCR amplicons are purified, sequenced (for example, using BIGDYE® Terminator chemistry (Applied Biosystems)), and separated on DNA analyzers (such as ABI PRISM® 3100 Genetic Analyzer). For each exon, a normal control sample is sequenced and used as a reference along with the publicly available sequence.

Variants identified in the inhibitor binding domain of the HMGCR gene are analyzed for association of a decrease in risk of cancer, such as colorectal cancer, with use of statin drugs.

### Example 7

### Identification of HMGCR Variants in Linkage Disequilibrium with rs12654264

This example describes the identification of additional HMGCR SNPs and their linkage with the rs12654264 SNP.

### Methods

Microsatellite stable (MSS) colon cancer cell lines (SW480, SW620, HT29, WiDr, and SW1417) were cultured in RPMI (supplemented with 10% Fetal Bovine Serum, 100 IU/mL of Penicillin, 100 IU/mL of Streptomycin, and 1× Non-Essential Amino Acid) at 37° C. in an atmosphere of 5% CO₂. The cells were trypsinized and washed with PBS twice. Cell pellets were collected and used to obtain genomic DNA (gDNA) using Puregene Genomic DNA Purification Kit (Gentra System).

HMGCR DNA spanning from intron 6 through exon 15 was sequenced in its entirety. Genotyping of the rs12654264 SNP was performed on 12 ng of each gDNA using Assays-by-Design SNP Genotyping Assay from Applied Biosystems (Taqman MGB probes, FAM and VIC dye-labeled).

RNA from each colon cancer line was processed and the cDNA was prepared using High-Capacity cDNA Reverse Transciption Kits (Applied Biosystems). Three Taqman assays as described by Medina et al. (Circulation 118:355-362, 2008) were used using approximately 300 ng of cDNA. Ribosomal protein LPO (RPLPO) expression was used to normalize the assay. The assay was run in 7900HT ABI Taqman Instrument.

### Results

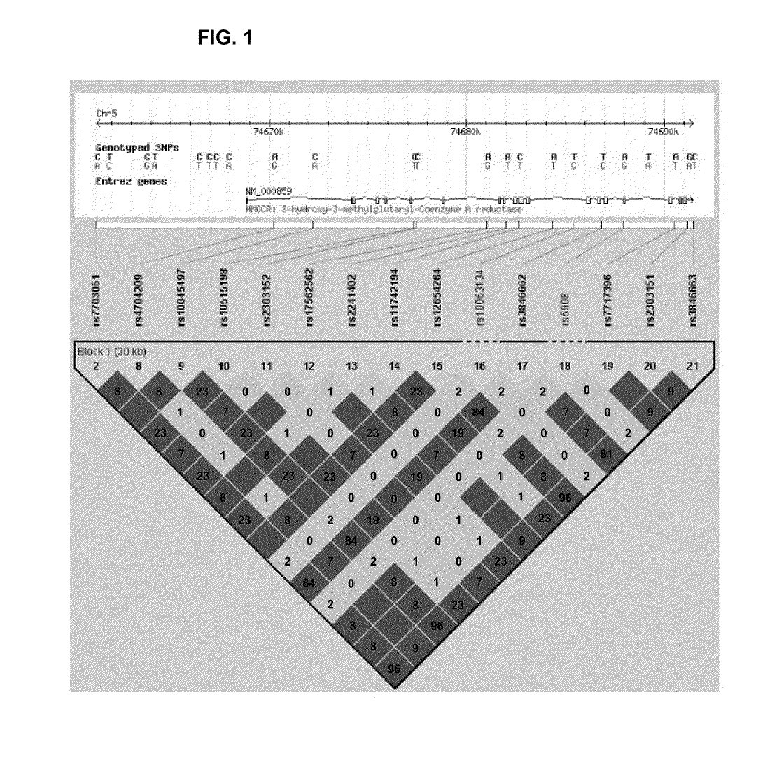

Twenty-one variants were detected in the region surrounding rs12654264, from intron 6 to exon 15 (Table 6). Ten were novel variants that had not been previously described. rs12654264 has high frequency (>40%) and the minor allele appears in 3 haplotypes with frequencies of about 10-15% each. Of particular note, one SNP (rs3846662) is in high linkage disequilibrium (r²=0.84) with SNP rs12654264 (FIG. 1).

TABLE 6
HMGCR variants in strong linkage disequilibrium with rs12654264

| | bp | SW480 (TT) | | S707B (TT) | | S823B (TT) | | |
|---|---|---|---|---|---|---|---|---|
| | (SEQ ID | SW480 | # of | | # of | | # of | |
| Location | NO: 1) | (TT) | clones | S707B | clones | S823B | clones | SNP |
| Intron 6 | ins | ---/TTC | 8/8 | ---/TTC | 6/6 | ---/TTC | 7/7 | rs17238456, |
| | 10,900 | | | | | | | rs45609440 |
| Intron 6 | 11,608 | T/G | 7/7 | T/G | 5/5 | T/G | 7/7 | rs6453131 |
| Intron 6 | Ins | -/A | 7/7 | T/G | 5/5 | T/G | 7/7 | rs11443896 |
| | 11,687 | | | | | | | |
| Intron 6 | 12,718 | A/- | 1/8 | | | A/G | 1/8 | New |
| Intron 8 | 13,391 | | | -T/T- | 1-2/5 | -T/T- | 2-3/8 | New |
| Exon 11 | 14,263 | T/A | 1/7 | | | T/C | 1/8 | New |
| Intron 11 | Ins | ----/ATTA | 8/8 | -/GTT | 7/7 | ---/ATT/ | 8/8 | Next to |
| | 15,013 | TTAT | | | | ATTA | | (upstream) |
| | | TATT | | | | TT | | of |
| | | ATTA | | | | | | rs35306582 |
| | | TT | | | | | | |
| Intron 11 | 15,398 | G/T | 8/8 | G/T | 3/7 | G/T | 2/8 | rs17238484 |
| Intron 11 | 15,505 | A/T | 8/8 | A/T | 7/7 | A/T | 8/8 | rs12654264 |
| Intron 11 | 15,798 | A/T | 1/8 | | | A/G | 1/7 | New |
| Intron 11 | 16,400 | C/T | 5/5 | C/T | 3/8 | C/T | 1/8 | rs10053643 |
| Intron 11 | 16,668 | TA/-- | 1/5 | --/AT | 1/8 | --/TA | 2/8 | rs17238498, |
| | | | | | | | | rs35823838 |
| Intron 13 | 17,986 | A/G | 5/5 | A/G | 8/8 | A/G | 8/8 | rs3846662 |
| Intron 14 | 18,427 | | | A/- | 1/8 | A/G | 2/8 | New |
| Intron 14 | 18,479 | | | -AA/AAA/ | 4/8 | -/A | 5/7 | New |
| | | | | --A | | | | |
| Intron 14 | 18,766 | | | G/A | 5/6 | G/A | 6/7 | New |
| Intron 14 | 18,811 | G/A | 5/5 | G/A | 6/6 | G/A | 7/7 | rs6882842 |
| Intron 15 | 19,525 | | | -A/A-/-- | 1/7 | A/- | 2/8 | New |
| Intron 15 | 19,660 | A/G | 8/8 | A/G | 3/7 | A/G | 2/8 | New |
| Intron 15 | 19,671 | C/T | 8/8 | C/T | 3/7 | C/T | 2/8 | New |
| Intron 15 | Del | CCGT | 8/8 | CCGTC | 3/7 | CCGT | 2/8 | rs17244897 |
| | 19674- | CCGT | | CGTCT | | CCGT | | |
| | 19,689 | CTGT | | GTCTG | | CTGT/ | | |
| | | CTGT/ | | T/---- | | CTGT/ | | |
| | | ---- | | | | ---- | | |

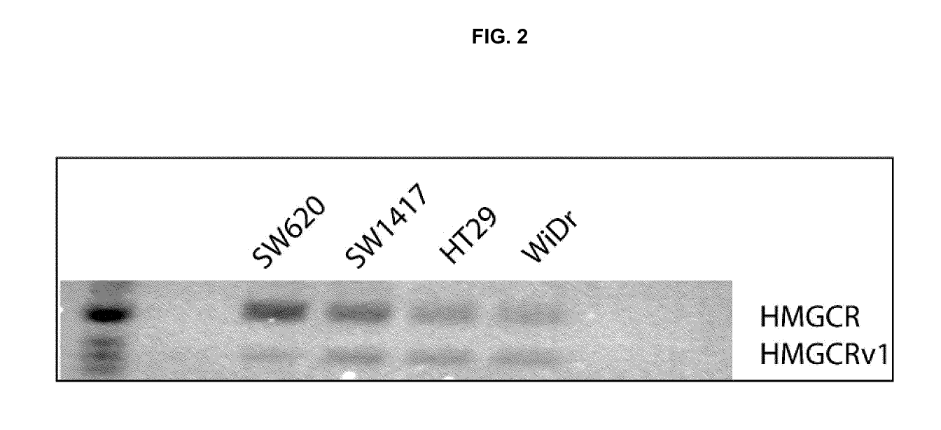

HMGCR SNP rs3846662 has been previously described to affect alternative splicing of HMGCR exon 13 (Krauss et al., Circulation 117:1537-1544, 2007; Medina et al., Circulation 118:355-362, 2008). Because rs12654264 was in high linkage disequilibrium with rs3846662, several microsatellite stable colon cancer cell lines were tested for the presence of the HMGCRv1 transcript, which lacks exon 13. Using RT-PCR, the presence of both full-length HMGCR transcript and HMGCRv1 transcript was detected in three colon cancer cell lines (FIG. 2). The genotype of the cell lines is shown in Table 7.

TABLE 7
MSS colon cancer cell line genotype

| Cell Line | rs12654264 Genotype |
|---|---|
| SW620 | TT |
| SW480 | TT |
| SW1417 | AA |
| HT29 | AT |
| WiDr | AT |

### Example 8

### Effect of rs12654264 SNP on Cholesterol Synthesis and HMGCR Splicing in Colon Cancer Cell Lines

This example describes the effect of the HMGCR rs12654264 SNP on cholesterol level, responsiveness to statin treatment, and HMGCR alternative splicing in colon cancer cell lines.

### Methods

The cholesterol level of each colon cancer line was measured using Cholesterol/Cholesteryl Ester Quantification Kit from Abcam (Cambridge, Mass.). Cells were plated into 6-well plates to reach approximate confluency of 70%. After overnight incubation, the cells were treated with 25 μM atorvastatin diluted in serum-free DMEM/F12 50:50 (supplemented with 100 IU/mL of Penicillin, 100 IU/mL of Streptomycin, and 1× Non-Essential Amino Acid) for 24 hours at 37° C., 5% CO₂. After treatment, the cells were trypsinized and counted to 1×10⁶ cells. The cells were pelleted, washed with PBS twice, and resuspended in 200 μl of pure chloroform with 1% Triton® X-100. The cell suspension was vortexed for 15 seconds, and centrifuged at maximum speed for 10 minutes. The lower (organic) phase was collected and air dried at 50° C., followed by vacuum drying for 30 minutes to remove the chloroform. The dried lipids were resuspended with 100 μl of the Cholesterol Reaction Buffer provided in the kit, and vortexed for 5 minutes vigorously at room temperature. 25 μl of the lipid was aliquoted to each well of a 96-well plate. This 25 μl aliquot represented the total cholesterol from 250,000 cells of the cell line used. In each well, 25 μl of the Reaction Mix provided was added and incubated for 1 hour at 37° C. incubator away from light. The Cholesterol Standard provided was used to perform the standard curve.

HMGCR transcript expression of the cell lines was determined as described in Example 7.

### Results

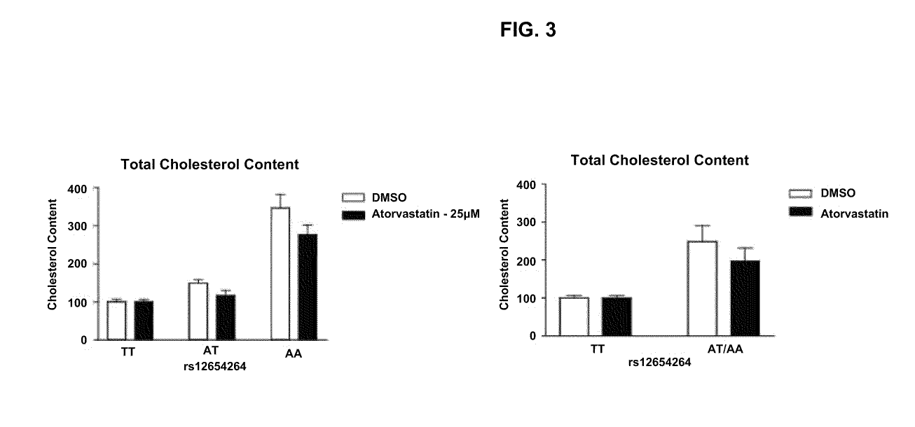

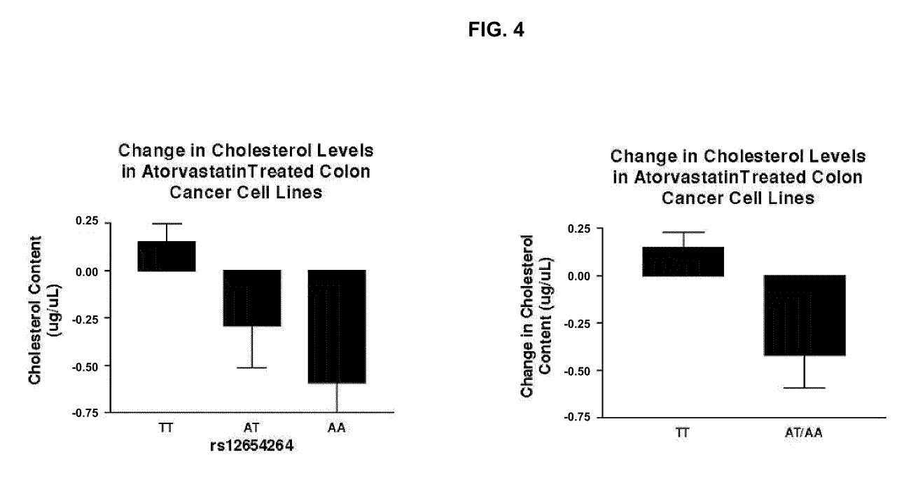

Colon cancer cell lines with both common and variant rs12654264 homozygous and heterozygous genotypes were analyzed for cholesterol content in serum free medium with no exogenous cholesterol. Cell lines with the rs12654264 A allele had significantly higher cholesterol levels (FIG. 3). When the cell lines were cultured with atorvastatin, cell lines with the rs12654264 A allele had a significantly greater statin-dependent cholesterol reduction as compared to cell lines with the rs12654264 T allele (FIG. 4).

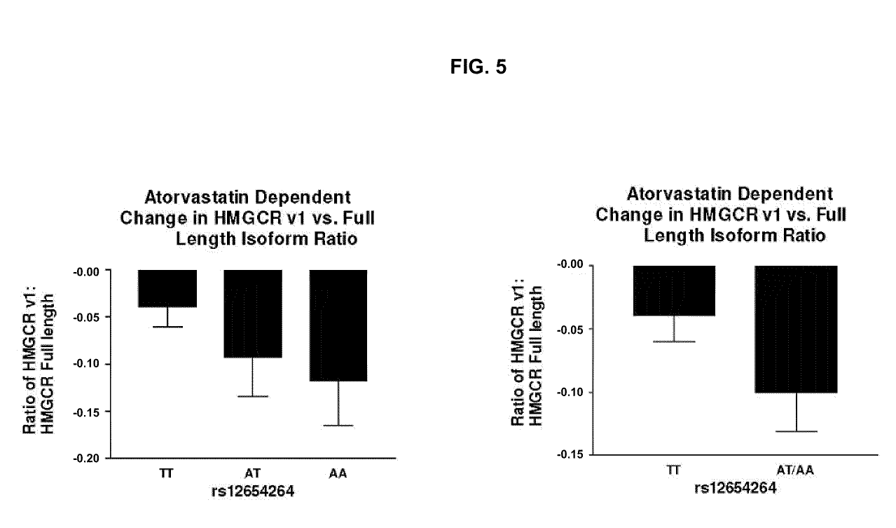

The cell lines were also analyzed for the presence of full length HMGCR transcript and HMGCRv1 transcript using a TaqMan assay. The rs12654264 allele was associated with a decreased ratio of the alternatively spliced HMGCRv1 transcript to the full length transcript (FIG. 5). The rs12654264 A allele and the rs3846662 A allele are tagging SNPs for decreased alternative splicing of HMGCR. This results in an increased amount of the full length HMGCR transcript with greater sensitivity to statin-dependent repression of cell cholesterol synthesis.

In view of the many possible embodiments to which the principles of our invention may be applied, it should be recognized that the illustrated embodiment is only a preferred example of the invention and should not be taken as a limitation on the scope of the invention. Rather, the scope of the invention is defined by the following claims. We therefore claim as our invention all that comes within the scope and spirit of these claims.

1. (canceled)

2. A method for identifying a subject which is a candidate for treatment with an inhibitor of 3-hydroxy-3-methylglutaryl coenzyme A reductase (HMGCR) to decrease risk of cancer, comprising determining in a sample from the subject the presence of at least one polymorphism in at least one allele of an HMGCR gene, wherein the subject has two alleles of the HMGCR gene, and wherein the at least one polymorphism comprises one or more of:
a) an A at nucleotide position 1176 of intron 11 of the HMGCR gene;
b) an A at nucleotide position 45 of intron 13 of the HMGCR gene;
c) a G at nucleotide position 224 of intron 5 of the HMGCR gene; or
d) a T at nucleotide 372 downstream of the termination codon of the HMGCR gene,
wherein the presence of the at least one polymorphism indicates that the subject is a candidate for treatment with an inhibitor of 3-hydroxy-3-methylglutaryl coenzyme A reductase to decrease risk of cancer, as compared to a subject which does not have the at least one polymorphism.

3. The method of claim 2, wherein the at least one polymorphism comprises an A at nucleotide position 1176 of intron 11 of the HMGCR gene and an A at nucleotide position 45 of intron 13 of the HMGCR gene.

4. The method of claim 2, wherein both alleles of the HMGCR gene from the subject are A at nucleotide position 1176 of intron 11 of the HMGCR gene.

5. The method of claim 2, wherein both alleles of the HMGCR gene from the subject are A at nucleotide position 45 of intron 13 of the HMGCR gene.

6. The method of claim 2, wherein both alleles of the HMGCR gene from the subject are G at nucleotide position 224 of intron 5 of the HMGCR gene.

7. The method of claim 2, wherein both alleles of the HMGCR gene from the subject are T at nucleotide 372 downstream of the termination codon of the HMGCR gene.

8. The method of claim 2, wherein the cancer comprises colorectal cancer, melanoma, breast cancer, prostate cancer, or lung cancer.

9. (canceled)

10. The method of claim 2, wherein the inhibitor of 3-hydroxy-3-methylglutaryl coenzyme A reductase comprises simvastatin, pravastatin, rosuvastatin, or atorvastatin.

11. The method of claim 2, wherein the treatment with an inhibitor of 3-hydroxy-3-methylglutaryl coenzyme A reductase comprises administration of a therapeutically effective amount of an inhibitor of 3-hydroxy-3-methylglutaryl coenzyme A reductase for at least five years.

12. The method of claim 2, wherein the subject is an Israeli.

13. The method of claim 2, wherein the subject is an Ashkenazi Jew.

14. The method of claim 2, wherein the sample from the subject comprises a blood sample, a buccal cell sample, a saliva sample, a urine sample, or a tissue biopsy sample

15. The method of claim 2, comprising:
obtaining a test sample of DNA containing an HMGCR sequence of the subject, wherein the test sample comprises genomic DNA, and
determining the presence of the at least one polymorphism of the HMGCR gene in the genomic DNA.

16. The method of claim 15, wherein determining the presence of the at least one polymorphism comprises using restriction digestion, probe hybridization, nucleic acid amplification, or nucleotide sequencing.

17. The method of claim 15, wherein determining the presence of the at least one polymorphism comprises an allele-specific oligonucleotide extension-ligation assay.

18. The method of claim 2, comprising:
obtaining a test sample of RNA containing an HMGCR sequence of the subject, wherein the test sample comprises RNA, and
determining the presence of the at least one polymorphism of the HMGCR gene in the RNA.

19. The method of claim 18, wherein determining the presence of the at least one polymorphism of the HMGCR gene comprises using nucleic acid amplification, Northern blot, or RNase protection assay.

20. The method of claim 2, further comprising determining a level of a C-reactive protein in a sample from the subject, wherein a subject which has an elevated level of C-reactive protein as compared to a control subject is a candidate for treatment with an inhibitor of 3-hydroxy-3-methylglutaryl coenzyme A reductase to decrease risk of cancer.

21. (canceled)

22. A method for decreasing risk of developing cancer in a subject, comprising:
selecting a subject by determining in a sample from the subject the presence of at least one polymorphism in an HMGCR gene, wherein the at least one polymorphism comprises one or more of:
a) an A at nucleotide position 1176 of intron 11 of the HMGCR gene;
b) an A at nucleotide position 45 of intron 13 of the HMGCR gene;
c) a G at nucleotide position 224 of intron 5 of the HMGCR gene; or
d) a T at nucleotide 372 downstream of the termination codon of the HMGCR gene, and
administering a therapeutically effective amount of an inhibitor of 3-hydroxy-3-methylglutaryl coenzyme A reductase to a subject which has the presence of the at least one polymorphism.

23. The method of claim 22, wherein the at least one polymorphism comprises an A at nucleotide position 1176 of intron 11 of the HMGCR gene and an A at nucleotide position 45 of intron 13 of the HMGCR gene.

24. The method of claim 22, wherein the cancer comprises colorectal cancer, melanoma, breast cancer, prostate cancer, or lung cancer.

25-26. (canceled)

27. A kit for identifying a candidate for treatment with an inhibitor of 3-hydroxy-3-methylglutaryl coenzyme A reductase to decrease risk of cancer by determining the presence of at least one polymorphism in an HMGCR gene, comprising at least fifteen contiguous nucleotides that hybridize to an HMGCR gene nucleic acid sequence, wherein the at least one polymorphism comprises:
a) an A at nucleotide position 1176 of intron 11 of the HMGCR gene;
b) an A at nucleotide position 45 of intron 13 of the HMGCR gene;
c) a G at nucleotide position 224 of intron 5 of the HMGCR gene; or
d) a T at nucleotide 372 downstream of the termination codon of the HMGCR gene.

28-29. (canceled)

30. The kit of claim 27, wherein the one or more primers or probes comprise one or more primers or probes having SEQ ID NOs: 8-16.

31-34. (canceled)

35. A method for identifying a subject which is a candidate for treatment with an inhibitor of 3-hydroxy-3-methylglutaryl coenzyme A reductase (HMGCR) to decrease risk of colorectal cancer, comprising determining by an allele-specific oligonucleotide extension-ligation assay in a sample from the subject the presence of at least one polymorphism in an HMGCR gene consisting of:
a) an A at nucleotide position 1176 of intron 11 of the HMGCR gene;
b) an A at nucleotide position 45 of intron 13 of the HMGCR gene;
c) a G at nucleotide position 224 of intron 5 of the HMGCR gene; or
d) a T at nucleotide 372 downstream of the termination codon of the HMGCR gene,
wherein the subject is an Ashkenazi Jew, and wherein the presence of the at least one polymorphism indicates that the subject is a candidate for treatment with an inhibitor of 3-hydroxy-3-methylglutaryl coenzyme A reductase to decrease risk of colorectal cancer, as compared to a subject which does not have the at least one polymorphism.


SEQUENCE LISTING (28 sequences)

SEQ ID NO: 1 - DNA, 24827 bp, Homo sapiens
ttcggtggcctctagtgagatctggaggtgaggcgggcggtgaccgagaagaggggcaggggcggcggggagcggggcggagatgggtgggagcggggtttgggctgtgttggtggcaattctggagcttccctcggccctgggaagtggctaccggcagctcctgcggacctggagggggctgcggttgcgctttgtcggtgtggcagctcggacccgcggggactgcaaggaatgtccttgaggcccggcaggccgagcggcggccggcatcagtgccggagtaacccggggtcccggggtgggcttgagaggcgggcggcggtctggcctcttcgtgactgcggtcatcatcggtggacccgcggggcgtagctgcgttcatcgtccctgttcagtcagagtaggcagtgctggctgcacggtcacgaaaatcggggcggaaagggtgtcaggcagggtgacctcggaggcccctggattcgagaaatgctaggggtctatggggctgtcgggccggcagctcgcagggcagacgggagaagcgcctgcatcccgggatccgggcattcacgcccaggaactgctgttcgttagcacctttcttttaggtgacgggaaagatctctgtaaatactgctgactaacttagaaccatgaaagaaccgtggattggtgtagatgtgtctggttatttacaggagaacggcttgagaggatgcggagcccaacgtggggattcgcacaatgactcaaaagattcttctccctctttttttttttttttttttggtaaggggtgtagtctccttggtgctgatattcttttaggaaaaatgtaccttggagatacaaatatagaacagttaatttctgcagtaggaggaggtcgatgtactccattcttaagtagtcgtttgaaatatttcaggatttgaggatattccttcttagacatggtcctgcagagtcgtaggaagcatttgtttctgggctatactaaatgtgcatgatttcggcgtcccacaaggtagagaatgtactttttgtttacttatgaatttattttcctctccatgcagtgaaaggcattttgcaatttagagataatctagcaaagacctggacagtactttggggataggctggattttgcaggcctgctttgttgtagtgttcttccagaacttatgataaacttggtggccaaaacaaatgggaattagtaacatgaaggttaaacaaaattgcacataggttacagcattaacatgaaaattgtcgggtatgtgggataaccatttcaagataagagtgtgagtatgcgtgcatgcacacactctttgtattcctttatggtctagtttctagactatagatagccacattatggaattacatggtctacttaggttgacccatgtgaaattgccagtatttaaaattcaactaacctacaaaaatagcagtttcatatgattccatttaacacttatctttgtcagagaactttggagctctagcttcataagtgaatgaagagaatagatatgacttaaaaaacaacatgtaatagaatgtgagctaattgggtatagaatccagatttcctgattgccagtgcaagattcgtttttttttttttttttttccttctttctggaataccagcttgagcagcagaatgctagatcttgttgggattgaaaatgactgccatataagacgcagttcttggcttcaaagagacaaaatgtacacatttattaatttgagaacactgtgagatagtatataatcaaatactggaatgtgtaatgcaggcggagtgctgagtaggaggaagtaagatgccaaaacatccttttctgcagttggccatggtaggtggcttctgtagcagattggattgacatttaagaattatccattcttttaaagaatctgtaaaagtttgcagcatactttttagtctgaataagtaaggcataccttgcctatttgtttctttaagtaaaattaatttttggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaattaatttttattgcagcattgcataaatactgtcaacttaatttgcaggagctatcctgtagcctgcttgtgacatttgcaactgctgatgaaggtggacgattgaattctatggcatgtaaatgctttgcgtaatcacagggcaacggggaggatgttcctgagaaatgagattgtttataattatggttacttttgacacaccctcataagattgctcagtttgtttccaaataggtggagttctctgtgtttaggggcggctgacagaatcatgtgtaatttttttccttgaatattgcaaagatgactttacacctaggaaaaaaagtcccagaattacaatgccaccataaaatcttttgagggaaaacataatgtgtctaatgtataattatgggtttatgatgttaaaccaatttgttcacaagttagtgttccttctgggttgcagctaagattttaactcaaatgaattaactttttaaaacaatgaatctgattgacataatgaggaataatctgagctgaagagtttttttttcagggaaagagggttagaaataattttgacctaattttgaaacatgtagttaatttactttctggaactttgatgctaattgatcctttagaacagttggtagagttgtgttttaaatgtaggaaagaaattatgtgatagaataccatgatgaaaatctagccttttagtgagtgagtcttttgtttataattaagtagagttcaaatctaagatttgattaaagatttccaatacttaaaagtccaagtacctgctaatttaatagaaagtattttgttgctgttgttcattttccaggatagttccaagttgactgtctctaatctgaagatctgaaatccgaaatgctccaaaatctgaaatttcttgagcaccaacctgacactcaaagaaaatgctcattgcagcatttggagtttggattttggattagggatgctaagctaataaatgtaatgcagatattacaaaattcaaaaacatccaaaatctgaaacgcttctggtcccaagcattttggataaaggatactcagcctgtatgtaaaatgtttctccttcccgtaccccattaataagttccctgaatgctctgcatttagccttaaactagagggcatgggttacatccatatctaggtgccttaaaaaaaagtcgtctttaggtctgtttcaagatgtgtcataaaatacatgctactatagcttaagaatttaagccagcatttgttgagcaatgctgtatatgagtgcaaggtgctggagggtaaattgatgaataagactcagggtggaaggttgaaaaagcatgagtaaacacaggcaggtacatgccacatttatattcctttaggtactgccagctttttaaaactgtgacccacagtaggaaatacacgttaatttaaaagccagacacacacttatataggtatagtttcttgaaacatagttttaagaaataatacctacccatactacctggcttacccaacagctattttttataatattgttttctattttattttatttaaaaatgtttgttcttaacgacaaaattgatgtcatgatgtaattgatgcagttgttgtggatcaacagtttgaaaactgtttagctagtggtgtggtatgtctgaaatgtagagaacatggagtgatgcagtgagagatgacacttggtatttgctttggttccttaaagctgctctttttttttttttttggtctttcccctaaccctttataaatagattaatgctttctttcacttacttgattgcctttttatagtctgtcttcaacagatgaatcacttttatttattactaaatgtgagattcatttgaattcttgctagagtggcagttaatgagctttttaaatctttttggttgatttttgcaggttactaatatgccttatcacaggcattgattttagtgaaagaagtaagacttgcaggtttatttgtggagaccaatggtaatagaagatttgttttcaaaatcagaatgtcataattattatttagcaaacaggaaatttagtcatggtagtcagtaaatcaaaatcttctatatgttcttttgggaatttttttgaatgctatatacagatacttcagccagtcatcctcttctagtactgttccagcttttgattagttgtgaaagtttggagcttttcacatctaatgcttttccattggtcttctagcactattgttgatgaatctgctagaagaaaagcagccaccacagcaaaattcttaaccctgttttattaatgtagaaacatctatcagaaaagactttttttctgttgacagcaatgttaggaaggtttattaaacatccttttgctagtgacattatgccatatgttctatggaatgaaaaagtacaagaggtccctgcccttgaggatcttatcaactaacatgatttatagcaggacactcaataagtggattcttgggtgtttaccttttgtgtaatcagaatgtagatgatgaagaagatacttaacatgcattttatatctaggtaattagaaaatgtgaatagctgtttctcacttgtgttttctgcttgattgctcttctacttgcaaggcttaggtaataaggtcgagatacttatctggtttgatcttaaatgtttgaattcatataatttttaagaaatggctgctttaaagttggttgccagtaagtaataaaggatttattgtttgagtgaagaagaaataacatagttctcttaattttataattattttccagaattataaggaacagtatcaaatagtcatatgtatgggacactgtgcatacaaagcagggtttatagcacacttttccttaaaatcttttcctaaaaatacaatgagctgtatactaagtgttcacccttgatattccttccaggatccaaggattctgtagctacaatgttgtcaagactttttcgaatgcatggcctctttgtggcctcccatccctgggaagtcatagtggggacagtgacactgaccatctgcatgatgtccatgaacatgtttactggtaacaataagatctgtggttggaattatgaatgtccaaagtttgaagaggttagtgaagttaatttgatactgactaaagtaaattacattttcaatttttgaagagcccttaagcccctatagggagcacataatttttaaaagttagagtaaaatatttattttagtattttggaacttacctcaaatttctgcttacatggaatgcactggaaatgctcttattttgctttgtctttacaaatgaatgtaattgactttatttgagaaatacatcttttataagtgactaatagtcaaaaatgattgtgggccgggtatagtgactcatgcctgtaatcccagcactttgggaggccaaagcaggagaattgtttgagcctaggagttcaagaccagcctgggcaacctggacaacatagtaagacccagtctttaaaaaaaaattaaaggccgggcacggtggctcatgtctgtaatcccagcactttgggaggccaaggcgggtggatcacgaggtcgggagatcgagaccatcctggctaacatggtgaaacccgtgtctctactaaaaaatacaaaaaaattagccaggcatggtggcgggctcctgtagtcccagctactcggaaggctgaggcaggagaatggcgtgaacccaggaggcagagcttgcagcgagccgagatcgcgccactgcactccagcctgtgtgacagagcgagattccatctcaaaaaaaattaaaaaattagctgggtgtggtagcatgtacctgtagtcctgctactcgggaggccatggcaggaggatcccttgaacctggaggttgaggctgcagtgaactgtgattgcaccactgtactctagcctgggtaacagcatgttaccctgtctcaaaaaaaaaaaaattgtggtgatgtattggcttaccacagtggtttgattaaaagttggatttaatttttgatttgtaggtttgatatttttattggacttgtattgtgcttacatttatgttctcatgactatataaatgaattacacatgcaaaataaaaattcttagttttgattacttattttaaaagtcaaagctaatggaatttccttttctttctctcctattaggatgttttgagcagtgacattataattctgacaataacacgatgcatagccatcctgtatatttacttccagttccagaatttacgtcaacttggatcaaaatatattttgggtaatagttttacatatttaacttctggtgtcatcagatttttgatttgctgtggaaataaacctgttcattcaaatcatgtatttggaaatatgtgtattgctgaaagtgtgctgcatgcattgaatagtttccttattattggcaattagacattctgaaatttgagaccttaataatttggagtaaactagtattatctcagaatgctgtgtgacactgttattatttgtgtagtaaaggataaagtttttttagtattgaacttttgtcattttaggtattgctggccttttcacaattttctcaagttttgtattcagtacagttgtcattcacttcttagacaaagaattgacaggcttgaagtaagtatttaaaacctaaatatactttctgtcaaaatacattttaaaaaacttttcttccccatgctgtaaaggtacattttcaaaagttaagaaaataaggggaaatttttttgtataattttactattagctaattttaataactattaacattttggcatatatccttttctactgtttttatacttaaagaaaatatctgatatcatatatattgttttataatttctttatgcttaataatagtttatcaacatctttccatgtcccttttttttttttttgagatggagtttcgctcttgtcacccaggctggagtgtaatggcacgatcctggctcactgcaacctccacttcccgggttcaagcagttctcctgcctcagcctcctgagtagctgggattacaggcacctgccaccatgcccatttaatttgtgtatttttggtagagactgtgtttcgccatgttggtcaggctggtgtcaaactcctgacctcaagtgatccgcctgccttggcctcccaaagtgctgggattataggcgtgagccactgtgcccggcctccatgtccttaatgttaaactaaattgtttgtaatggctatatgtattctcttctgtctaaacgtcttgtagtttattaatcatctaatgttggactgttaggttatgtaattttttttattaataacaacactgtgatgaatgtctttgtaacgaaatttttgttcatatttgtaatcattttcttaagatacattcctagaagtgagacagtggttttgctttttttagagctttgcttttttttttttaagagctttttagtgctattattgccaatttagtttacagaaagtttgtttagtttatccttccacaggtagtgatcaatgaaaatttttatatattccactttttttccctaatggtaatccaaggagatattttttactaaggatgatactttgatacaaaattatcaaaaggtgtttaaatgtaaatatacttacattttaacattaaaaatatttttaacaaatattttgagcaactactatgtttagctttgaggatgccaaagaaatatagggtatacttttttgtctgcagaaaggtacacatactacaggatcatacagtatggagggggaaaggttttgttctaaaaagaatttttttaaaatcatactttttcccgttaaatttatctgatcattttgttcttttccagtgaagctttgccctttttcctacttttgattgacctttccagagcaagcacattagcaaagtttgccctcagttccaactcacaggtaagtattatttataagataggaatgtgaatagtattccttttttcagtttacattaataggaaggattaatagcgtttcttcatagcacaagatttaagaattgcccaaagttttaagtttaattctcaagtcccaagactggtctccataagtgccccaggaacagtcccctgtatctaacaactcaattatgattctgtagctactggaatttggaattgcccccatttttctttttgaaagttttcagaacttatggtaataataatttttgggtaaataagagtattttcctagtgaaacacatcagagagcagaacaagatctaatggaagagaaacccagggtagattgattgattgattgatttgagatgtagtctcactctgtcacccaggctggagtgcagtggcgagatcttggctcaccacaaccttcgcctcctggtttcgagggattcttctgcctcagccacctgagtagctgggattacacatgtgcaccaccatccccggctaatttttgtgttttcagtagagacggagttttgccatgttggccagactggtctttaatttctgacctcaagtgatccacccgcctcagcctcccaaagtggtgggattataggtgtgagccactgcacctggccagatttatttttaaaccccttaaattaacactggcattatttcctattttaagtttcatcttaatgttcattagtcactttgaagaacctaaccattttaacaatattatatcagagtatcttgagtataaaatagcacacaattctgccctgattatttgttgatgaaagtgtgtgatcatttgaacatattaacaaggaaataagtttggttttattaatactaatatgatctcataaaatttgcaactaagcttctgtcatggtaggtaaaaatatagaatgttgtaggatttaattatgtgtgatttaaatgacagagttaatgcactacctcaaaggaatattcctaacaaatatcttattcagtggaaagggaatcagggcactatatgttcttttttaaaaatttggctgggcgcagtggctcacacctgtaattccagcacttcgggaggccgaggtgggcagatcacctgaggtcaggagctccagaccagcctggccaacctggtgaaacccagtctctactaaaaatacaaaaattagctgggcatggtggcgggtgcctgtaatcccagctactcgggaggctgaggcaggagaattgcttgaacccaggaggcagaggttgcagtgaaccaagatcacaccattgcacattgcactccagcctgggaaacaaagtgagactacatctcaaaaaaaaattttttaaaatcctttatattacaatcatactttgtatcttgaatacctgttagttttatcatattgtatattttactctttgaatagtaatttgatattaatataagccataggatgctctaacatttaaaaaagttgttctgtcccttgccttcattgatatgtttgatctgttttaggatgaagtaagggaaaatattgctcgtggaatggcaattttaggtcctacgtttaccctcgatgctcttgttgaatgtcttgtgattggagttggtaccatgtcaggtttgtaagcaatttttgccatattttaaaataggtatgtcgctaaagaggaaaaagaacatcttggatttgtattatttattgttaaattctgacttttaaattactcttaaaattttttattatattagtgtgtgggtatatctggcctgtttgctttggtggaaacttagcagcaggttactgatttatttttcactcctgccatcctctctttgtgttccactttgtgatatttttattcatttgatttcttttgtttgtttttttaaccaacattgcattccaaggttggtctggaaaacactttccagcccctgctgttacttagactcaattggtgacttggttctttgtgttttaattatcatgtagggagaaaagagtcaggaattggacagatctgagtaagattcacctaataaggtgattggaaatatttaatccgatagtaagcagtacatctcagcaagtacccagcattagcttcaacacatgtttcttcattcttaattggatagtagagaattagatattaatatcaaacataggacttcattatgtagaataaaaattacagactggcatggaaatttgccagaaggaatatattacgctgtgtgaaggtctcctaatcattaggattatattttatggctttcttataaagaccatacttggttaactactgtgtatttcctggtaccttagagccctcaatgagatgcctcattctttcagaagtaaattatctaacttttaagggggtaaattattgtcattagtttaggcctcttcagtagatagtatattaaaaatgtaatattgcttctaactttctgagagcaaattattatttatttattttattcaacaaagattttatttagcaaataaaatattttttaattttaaaaatttacttactttaaaatttacttttgggagtacaattttatgagttttgacaatgacatagtcttgtaaccaccactacactcaagaaaaaatgttgatcaggtgaaaatgtacttgtttgattttattcaagcaaaagaatatatgagaaggggattcgccaggaagtaatagttgggccatgaaaccatgggattattcattgaaataataatgatgtggaaagcagctgggcgatgagttttgtcatgatatggtcagagaagtagacaaaggaagtgattagatttgtggttggagaagacagtgagacatttacagctatacagaaatggtataattagagatttgggttacggtaaaaaaggacagatttaacacatattaacttggaagtaaccctttgaaaaagccagtacaaaaagaagaaagtcttagcttcttggatgtctgtggattggtgctgtttctttgtgtgtagagagacttatctggggacacagtaaggaagaatgcctggagagcatatggcaacaacatgagtgaatattgtgcagagagaaatagtagatagtcaagtcatgaaagcattggtcaagtatggttttaatggcctcaagtttgaaaagattatattcagctactatttaaaatgtagtttgatttataatgcttttgttattgtgattttgtagtctcttgaatttccacatttgagggtaatcgtggaattaaaaaaatacaatatatgcttgttctagtcagtcaaatgatatcttaaaatttaggtaataaaatacaactttgagtttttgtgagcagtttttataggacaatttaaatttatgtttcaaagcattgggaattaaaaaaatacaatatatgcttgttctagtcagtcaaattatatcttaaaatgtaggtaataaaataaaactttgagtttttgtgagcagtttttataggacaattcaaatttgtgtttcaaagcattgttatgtttgagcgacatcagaaatatgttttatattatactcaagtttatttgtaaagtgagggagaaggatttggtggtcatctaaggtctcttctagcctctataattctgttttctatttgagatcaccaaatgataccttaaacagttgtagtacaatatgtttgttaatttttagtcttaagttggcattttgtattatatcattttccaaacagatcaatgaaataaccaaaattataagaacttcagtaggcatcatagaggttatattgaaattagagtttgttggtacacaaaaaatattattttgaccttatatcaggactggcataactggcaggatattctacttatataaaaaatccttggttaattggcaaattgcttttctcctaacaagtgggattagatcaatagtgtcactggggttttgctctgttagagtagcctgtcctctccttatttaataataaggcactactgcttgacaaaaaggaattggaaacacatatgttttatcaattgtgtattaaacactaccattctgcctggcattgtgatatggggacatgagagaaggcaagagctttgctcttgagggacttagagttctgttgttattcctgctgtttcctaaggcttggcatcacctctaagttgctaattctatttccagtaagtggcaaggagcttaatgtactaatattttcatgttttgtccacctgcaggaagacaaatatatccttgtgatatatgcagcataaaaaataacgtagactttactagttgtatctttaatttttctctaaccaggggtacgtcagcttgaaattatgtgctgctttggctgcatgtcagttcttgccaactacttcgtgttcatgactttcttcccagcttgtgtgtccttggtattagaggtaagaccaattcttacatatggcactagtagaagagtaagatttctgcttacacagtttacctaaacagaatcaataccttctaatgtcacactgacttaatttgtagctttctcgggaaagccgcgagggtcgtccaatttggcagctcagccattttgcccgagttttagaagaagaagaaaataagccgaatcctgtaactcagagggtcaagatgattatggtaatgacatggttttcttcttcttttagtatcctcagttccaatctcattatttttaagatttctttttttctacaattttggcccattcaatgatattgcacccccttcttcctttttttcttaatgtgttcatttctttgaggctcctggtctttattagcccctctctcctaaacagactttttaagttcccccaccttatctctcgttgaaagcctgttctttggggtgttttcagtagttcagtggggtcactactttagttagttgcatagcaagcttggggctttttttttttcatggtaaggggaagctatgagagataatgtctggctgtccagttgctagggataagaaatttaagttctattgatatgcagaggatacattactttaaaaattttatttcagtctctaggcttggttcttgttcatgctcacagtcgctggatagctgatccttctcctcaaaacagtacagcagatacttctaaggtttcattaggactggatgaaaatgtgtccaagagaattgaaccaagtgtttccctctggcagttttatctctctaagtaagttaattgaaatctactttgtgatatattaatcataacactctatgctaatgtaagtttagattgtgtcctttacatttctgaataagattttaatttgctttcttttatttagaatgatcagcatggatattgaacaagttattaccctaagtttagctctccttctggctgtcaagtacatcttctttgaacaaacagagacagaatctacactctcattaaaaaaccctatcacatctcctgtagtgacacaaaagaaagtcccagacaattgttgtagacgtgaacctatgctggtcagaaataaccagaaatgtgattcagtagaggaagagacagggataaaccgagaaagaaaaggtaacttgttattctcttcgctttcaatccttcattgctttgtcaaaaagtagtctgttttcaaaattatgtgccgtgttgtgagatttcttttgatttcttgaacagttgaggttataaaacccttagtggctgaaacagataccccaaacagagctacatttgtggttggtaactcctccttactcgatacttcatcagtactggtgacacaggaacctgaaattgaacttcccagggaacctcggcctaatgaagaatgtctacagatacttgggaatgcagaggtgaggatgataacataaactccaatgtggcatttttcattacaaaggagctttttcaaggaagaaaaatctagtatctgctgaacactacagctaagttctgggcacggtgtaacatgactaacagatactatcttctttctttatttcacacaaccttgagaggtaggtacaattatctatttttcagatgagaacattgaggctccaatatgtttaatttcccaaagtagtacctctaggaaatgataaagctgatagcagggtccaagattttctgactccagagtcaaaactctttctagtttattactgtttatcatagagatgagtgactactgtattctcataggtgtgttgaggcctagaaagagtttaacacagagacaagtttcaaagatagaagaaagtttgtttttgttttgttttgtaagcttgatacccatgaggaagtttgcttttctttctgacatttgaacaggaccttctgcctacatgaccatatgaatctactcatgctttcatgcaaataatcatgttccatccatgtctgcttaatatggttctttcttttaaatatatgttaatacgtttattggtaaaatgcaattttttgccagctttattgaggtatacttgacaaaattttattttattattattattattattattattattattgttttggagacagagtgtcactctgtcacccaggctggagtacagtggcaccatcttggctcacagcaacctccacctcccaggttcaagcgattcttgtgtatcagcctcccgagtagctgggattacaggcatgcgccaccatgtctggctaatttttgtgtttttagcagagatgggatttcactatgttggccaggctggtctcaaactcctggcctcaagtggtctgcctgccttggcctcccaaagtgctgggattacaggtatgagccactgcatgcccggcctgcattcttcaatcatatgctttttagctaaacttagtcattgttctttaaatacagttcccaactctgggttcctattcttgtcatctgacatgtctccccctctccaggtgttcacacctttagggcaaatgctagcatctctatgaaacctacctcaattccgccaaacagaagtaacctttcttttctgaagcatccattatatagagactgtgcatttttaatggcagtcgtaccttgttgcttatatcacatttacatgaatatctccattattagacatcgggctcttagatggctaagtccatgtcttgagagggccttggatttcataggtgctcagtaaacttactaagagaatgaatgaatttgcattgataatacataaaccagctaatgtttatcttactattcttttgaaaaattaggctaatattagtgtttgcaaagaagatccttgaataaacccacaaaatggaattactggtaccagttttgtggagggtggtgattgtgagtgtgtgtgtgtgtatgtgtatgtatgtatatcaccaccacaagcagagtcaagagacagctaataaaccaagagaaaatattggcaatttatatttaaggctaaagggctaatctctaatatataaagagctcctacaaattgatgagactaaatttttatctaatttttaaaaagagcagataatcagtctattgtcaggaaaaaagtacatatggctcttaaaaatgtgaaaaggtgattaacttcatcctaagagaaatgcaaattaaaattgtacttgagatattttcactcatcagattggcaataatcccaaagtttaataacatactctattgaggttgtagggaaaggtcttttcttgtttttttgggtttttttttggtttggtttttttttttttgagaaggagttttgctattgttgcccaggctggagtgcaatggcatgatcttggctcaccgcaacctctgcctcctgggttcaattgattctcctgcctcagcctcccaagtagctgggattacaggcatgcgccaccattcctggctaattttgtatttttagtagagacggggtttctccatgtttgtcaggctggtctcaaactccggacctcaggtgatccgcccgccttggcctcccaaagtgctgggattataggcgtgagccactgcccccaggctggaaaggtcttttcttacattggtggtgagaaggcaattggaccgtgtttattcaaattaataaatttgtaaaaattataaaaatttataagattgtaaatgttcataccctttggcccaacacttgtgtgaatatatacccatatgtgtgcgtgtatatatatatatatgcatatttataatcatgcctaaaaacatctctggatggatacacagtgaaacagtggtacctatttatgtgtaggtagcaaagaactgggcagatggtagacagggatgggatgaagactttttgttatatctcttttaatatttagtttttaaaccatgtgaatatattgcctgttcaggatatcaaaaatagcaagtttactagagtttaaacaatagctgttatatctgacacacacttgaaaccttgctaaattcacatttatttttctttttatggtgtattagtgcaagcctgtctttgtattgtaaaatctaatgatacggtatttatattatttttgtttggcattttttgcattaaatgaattattttgcagaggtatcttttaattaaaaactacagtgatttaatttaaaaattacattattttagcttagcattgtttgtattaaatggtttataacatgaaatacagtccttcaagtcttctgtttcatctctctctctgaccacaatttcattttttttctccatttctttagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtatgttattttctcgattaagagagatttgctttgtatgtttttaatcttttttcttgattagtttcatatatgtacatagttttataaaacattttccttttaaatcattttatcctaattttttattctgcttatgatgtaggtcatagaaattaaaaatatatttcctgcttttatagtcattactcaaagattttagtattttaaacactttttaaaggtgaattaaacattttgtttaaaaagaatacatactaaaggattaagtttgaagatagttatactgacaagctgagataaaattttgtgcatttactatatagattttcatttggtgcctgactttaccttttaggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtgtaagttggcatttatatatttgccagtttaaaaatacatcataagtaaggcaatgagaagagttttaaggacaattagtgataccttttgggtcaagcatgagcatttttgggtaacatgtgcttgcttctctaacatatactgtgtagcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcaggtgtgtgagtggatttgtatgtacagttatatctatttgtttattttagaaccagtgtcattttctgtgattaccaaacataattgttaacatattacctgctaaagagcacataacagaatatcaactttaaagccattcatttaaaatgagtaatatttatgcttggttggggggaaaaaaagaatgttgattcaaatgaatagctccacagaggtaaattagtaagaaaaaaaaaaaagtgtgagctagtaattttgattagatgttactttgcctaggagagaactgtcttagaaaaaaagatttttcaaataggagagaaatattagtataataagacttacttcaaataaagaaaattaataaagtagcataatcaacacaaatgataaccatagtatagttcaagctaacacatttgtttttatgtgaactgtgtaagtttattaagaaataattgtgactgggcgcagtggctcacgcctgtaatcccaacactttggggaggccaacgcgggcagatcacttgaggccaggagttcgagaccagcctggccagcatggcgaaaccctgtctctactcaaaatacaaaaattggctgggcatggtggcccgcgcctgtaatcccagctactcgggaggctgaggctggagaatttcttgaacccgggaggtggatgttgcagtgagccaagatcaagccactgcactccagcctgggccacagagtgagactccgtctcaaaaaaaaacaaaaaacaaagaaataataataataaaagaataaaacacagtctttgcatcttttatttatagatttgcacgtctacagaaacttcatacaagtatagctggacgcaacctttatatccgtttccagtccaggtcaggggatgccatggggatgaacatgatttcaaaggtaagtgtaggcagagtatctgaaagtacttttattaaaatgaaagtacttttataaaaaacaaatcaggcatttgttgattggtattccttacatttgatagatttaatttagacttggcattacacatatcctgagtttacttaggactgggaacaatcttagtattgacatttcaaaactttatccagtcaagagaccacctttgaagctgacctcttcaagatttgtctttaagaacaccaatataatttcagtactttgggaggccaaggcaagaagattgcttggggtctggagtttaagaccagcctgggcaacacagtgagatgctatctctacaaacaataaaaaaaaatagttgtgcatagtggcacacacctgtggtcctcgctacacggtaggctggggcaggaagttagcttgagcctaggagcttgaggccagcttcggcaatgtagcaagaccctgactccattcatccatccgtctgtccgtccgtccgtctgtctgttttcaatttaggatattttctgcttatgtggggtttacagcctaataaacctatcaaaagtcaaggagcatctgtataagctttaaaattgccaataaacagtatggcagttcaacaacttaaacatgaaattaccatatgatccagcagttgcacttccgggcatatacccaaaagaactgaaagcagttctcagagagatatttgtacagctatgtttgtagcagcatttttcataatagccatgaattggaatcaaccttagtgttcgttgttggatgaatggacaacaaattagatgtatttgtatgtgtgtgtatgtatatgtctatataaaatggaatattattcagcattaaaaaggaaggaaattatagcacatgatacagcatggataaacattgatgacattatgctaaatgaaataaaccagtcacaaaaagacaaatactgtatcattccacttgaatgaggtgtctagaatagtcaaattcatagagacagaaagtagaatggtggttatcatgggtttggggaaggggaaaatgggtggttgttatttagtaagtacagagtttttgttttgtgagatgaaaagagttctgggagcaaagacatggaaccaacccaaatgcccatcaatgatagaccggataaagaaaatgtggcacatatacagcatgcaatactgtgcagccataaaaaaggaatgagatcatgtcctttgtagggacatggatgagcctggaaatatcattctcagcaaactaacacaggaacagaaaaccaaacaccacaccttttcacttgtaagtgggaattgaacaatgagaacacatggacacagggaggggaacaacacacaccgaggcatgtcggggggttgggggcaaggggaagagcattagggcaaatacctaatgcatgtggagcttaaaacctagatgacaggttgataggtgcagcagaccaccatggcacatgtatacctatgtaacaaacctgcatgttctgtacatatatcccagaacttaaaaaagagttctggggatttgttgcatggcagtgtgaatatacttaccactgtacacctaaaaatgggtaggatggtaaattttatgtgtttcaccacaataaaattatgtttttaattgccaataaatatgttttggattattgaggtatgagttgttaattgaattaatgccatttaattggaagtcattgggaagcttatttgtattttatgtaatataaagctcaacgacataagagtcgaatcacaagatcaactcttatgacccacttctctttacagacttcattacttggggttttaggtaacagctgaataactagaagatggaatttattactcattccccagtgacatataaatatgagtatttttattgaatatttttaacattatcatcttttatctacatctttttcaacaatgactacaaagaacttggttttcataccgagaaagaaacacattaggaatttatacctgaccaagaaagtaattgtgatggttaacaaaacagctgttacttttgtgctcagtttctttgaagtatagttattatacttagaaatatatcatagatacaaattcttctcataaggatgaaggatgaatatttcatgcagtttaatatgtgtgtgttttgggttcttgttaacagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtgagtgactggatggataatttatcttttttattttgtaatctttaattgtatttaaaaatgggggaaaggagtattaacattttaaataaagttaaatatatgggacagtgttttccatcaaagatgactgttgtaccttgcccatctgtctgtgtgtatcatccataggaacaaactttactgattttttttaattttttttattttttaatggaggacagggcttaaatggggccacatctaaactttgttttctggaggttcagaaagatagatttgggtaacattcccctgaaccttctggaggaacatctaaatgtacacagctctgttttgtaggtattaaagactaccacagaggctatgattgaggtcaacattaacaagaatttagtgggctctgccatggctgggagcataggaggctacaacgcccatgcagcaaacattgtcaccgccatctacattgcctgtggacaggtgagctctccagcctccacttctcttgtgttacgtctttctaagtgaaagaagtatattgtatattttttcttttcttgtttccaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcaggtatgatgtatcaggcatagagtccacaagcctagttctgactctctgggtttctctttctatctgagactatgtatcactcacctctattttaattggtcttttccaaactcttttgtcatatcagcctaatccattgtgtccaaataagcatgtttaagcttatgcttagataagaaagtagatgaagagagcaaatgaatgttcatctactgagttaagggtactgccagtcaggctgtgaatattatgttagctatggtattatgcactgtcaggtgtggctgtcaagtcttggaaagttagtgcttccagtggagtctagttctattctgatgccattacagttgccctgtttttagttgatttagtaagaaattggtcatgattttaagggtgaatcttgttgtgtctctccctggctacagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtaagactcaaagatatatttaacatgttccccctatacttcaaaaaatatgcagtgtaaaaacttactattcatctactgtagttccaagttaaaattctacactcctgatatttatatattgctactttgtcattttctaccataggtcgaagatcaatttacaagacctccaaggagcttgcaccaagaagacagcctgaatagcccgacagttctgaactggaacatgggcattgggttctaaaggactaacataaaatctgtgaattaaaaaagctcaatgcattgtcttgtggaggatgaatagatgtgatcactgagacagccacttggtttttggctctttcagagaggtctcaggttctttccatgcagactcctcagatctgaacacagtttagtgctttacatgctgtgctctttgaagagatttcaacaagaatattgtatgttaaagcatcagagatggtaatctacagctcacctctgaaggcaaatataagctgggaaaaaagttttgatgaaattcttgaagttcatggtgatcagtgcaattgaccttctccctcactcctgccagttgaaaatggatttttaaattatactgtagctgatgaaactcctgattttgtagttaatttattaagtctgggatgtagaacttcaagaagtaagagctaagttctaagttcatgtttgtaaattaatacttcatttggtgctggtctattttgattttggggggtaatcagcattattcttcagaaggggacctgttttcttcaagggaagaaacactcttattcccaaactacagaataatgtgttaaacatgctaaatagttctatcaggaaaacaaatcactgtatttatctccgcaggctatttgttcagagaggccttttgtttaaatataaatgtttaaatataaatgtttgtctggattggctataacatgtctttcagcattaggcttttaagaaacacagggttttgtattctttactaaagatatcagagctcttaatgttgcttagatgagggtgactgtcaagtacaagcaagactgggaccttagaaatcattgtagaaacacagttttgaaagaaaaataccatgtctctaagccaactttaattgcttaaaagacatttttatttagttgaaaaatctagttttttttgtaaactgtatcaaatctgtatatgttgtaataaaacttatgctagtttattggaagtgttcaagaaataaaaatcaacttgtgtactgataaaatactctagcctgggccagagaagataatgttctttaatgttgtccaggaaaccctggcttgcttgccgagcctaatgaaagggaaagtcagctttcagagccagtgaaggagccacgtgaatggccctagaactgtgcctagttcctgtggccaggaggttggtgactgaaacattcacacagggctctttgatggacccacgaacgctcttagctttctcagggggtcagcagagttattgaatcttaattttttttaatgtacaagttttgtataaataataaagaactccttattttgtattacatctaatgcttcaagtgttgctcttggaaagctgatgatgtctcttgtagaagatggactctgaaaaacattccaggaaaccatggcagcatggagagcctcttagtgattgtgtctgcattgttattgtggaagatttaccttttctgttgtacgtaaagcttaaattgcttttgttgtgactttttagccagtgactttttctgagcttttcatggaagtggcagtgaaaaatatgttgagtgttcattttagtgactgtaattaatatcttgctggattaatgttttgtacaattactaaattgtatacattttgttatagaatacttttttctagtttcagtaaataatgaaaaggaagttaatacca

SEQ ID NO: 2 - DNA, 4471 bp, Homo sapiens
  CDS (51)..(2717)
ttcggtggcctctagtgagatctggaggatccaaggattctgtagctacaatgttgtcaagactttttcgaatgcatggcctctttgtggcctcccatccctgggaagtcatagtggggacagtgacactgaccatctgcatgatgtccatgaacatgtttactggtaacaataagatctgtggttggaattatgaatgtccaaagtttgaagaggatgttttgagcagtgacattataattctgacaataacacgatgcatagccatcctgtatatttacttccagttccagaatttacgtcaacttggatcaaaatatattttgggtattgctggccttttcacaattttctcaagttttgtattcagtacagttgtcattcacttcttagacaaagaattgacaggcttgaatgaagctttgccctttttcctacttttgattgacctttccagagcaagcacattagcaaagtttgccctcagttccaactcacaggatgaagtaagggaaaatattgctcgtggaatggcaattttaggtcctacgtttaccctcgatgctcttgttgaatgtcttgtgattggagttggtaccatgtcaggggtacgtcagcttgaaattatgtgctgctttggctgcatgtcagttcttgccaactacttcgtgttcatgactttcttcccagcttgtgtgtccttggtattagagctttctcgggaaagccgcgagggtcgtccaatttggcagctcagccattttgcccgagttttagaagaagaagaaaataagccgaatcctgtaactcagagggtcaagatgattatgtctctaggcttggttcttgttcatgctcacagtcgctggatagctgatccttctcctcaaaacagtacagcagatacttctaaggtttcattaggactggatgaaaatgtgtccaagagaattgaaccaagtgtttccctctggcagttttatctctctaaaatgatcagcatggatattgaacaagttattaccctaagtttagctctccttctggctgtcaagtacatcttctttgaacaaacagagacagaatctacactctcattaaaaaaccctatcacatctcctgtagtgacacaaaagaaagtcccagacaattgttgtagacgtgaacctatgctggtcagaaataaccagaaatgtgattcagtagaggaagagacagggataaaccgagaaagaaaagttgaggttataaaacccttagtggctgaaacagataccccaaacagagctacatttgtggttggtaactcctccttactcgatacttcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagcctgaatagcccgacagttctgaactggaacatgggcattgggttctaaaggactaacataaaatctgtgaattaaaaaagctcaatgcattgtcttgtggaggatgaataaatgtgatcactgagacagccacttggtttttggctctttcagagaggtctcaggttctttccatgcagactcctcagatctgaacacagtttagtgctttacatgctgtgctctttgaagagatttcaacaagaatattgtatgttaaagcatcagagatggtaatctacagctcacctctgaaagcaaatataagctgggaaaaaagttttgatgaaattcttgaagttcatggtgatcagtgcaattgaccttctccctcactcctgccagttgaaaatggatttttaaattatactgtagctgatgaaactcctgattttgtagttaatttattaagtctgggatgtagaacttcaagaagtaagagctaagttctaagttcatgtttgtaaattaatacttcatttggtgctggtctattttgattttggggggtaatcagcattattcttcagaaggggacctgttttcttcaagggaagaaacactcttattcccaaactacagaataatgtgttaaacatgctaaatagttctatcaggaaaacaaatcactgtatttatctccgcaggctatttgttcagagaggccttttgtttaaatataaatgtttaaatataaatgtttgtctggattggctataacatgtctttcagcattaggcttttaagaaacacagggttttgtattctttactaaagatatcagagctcttaatgttgcttagatgagggtgactgtcaagtacaagcaagactgggaccttagaaatcattgtagaaacacagttttgaaagatttttaccatgtctctaagccaactttaattgcttaaaagacatttttatttagttgaaaaatctagttttttttgtaaactgtaccaaatctgtatatgttgtaataaaacttatgctagtttattggaagtgttcaagaaataaaaatcaacttgtgtactgataaaatactctagcctgggccagagaagataatgttctttaatgttgtcaggaaaccctggcttgcttgccgagcctaatgaaagggaaagtcagctttcagagccagtgaaggagccacgtgaatggccctagaactgtgcctagttcctgtggccaggaggttggtgactgaaacattcacacagggctcttggatggacccacgaacgctcttagctttctcagggggtcagcagagttattgaatcttaattttttttaatgtacaagttttgtataaataataaagaactccttattttgtattacatctaatgcttaagtgttgctcttggaaagctgatgatgtctcttgtagagatgactctgaaaaacattccaggaaaccatggcagcatggagagcctcttagtgattgtgtctgcattgttattgtggaagatttaccttttctgttgtacgtaaagcttaaattacttttgttgtgactttttagccagtgactttttctgagcttttcatggaagtggcagtgaaaaatatgttgagtgttcaaaaaagtgactgtaattaatatcttgctggattaatgttttgtacaattactaaattgtatacattttgttatagaatacttttttctagtttcagtaaataatgaaaaggaagttaatacca

SEQ ID NO: 3 - protein, 888 aa, Homo sapiens [sequence omitted]

SEQ ID NO: 4 - DNA, 101 bp, Homo sapiens
  misc_feature (51)..(51): n is g or a
ctgtatctaacaactcaattatgattctgtagctactggaatttggaattncccccatttttctttttgaaagttttcagaacttatggtaataataattt

SEQ ID NO: 5 - DNA, 101 bp, Homo sapiens
  misc_feature (51)..(51): n is a or t
cctacctcaattccgccaaacagaagtaacctttcttttctgaagcatccnttatatagagactgtgcatttttaatggcagtcgtaccttgttgcttata

SEQ ID NO: 6 - DNA, 101 bp, Homo sapiens
  misc_feature (51)..(51): n is t or c
tgaaattcttgaagttcatggtgatcagtgcaattgaccttctccctcacncctgccagttgaaaatggatttttaaattatactgtagctgatgaaactc

SEQ ID NO: 7 - DNA, 101 bp, Homo sapiens
  misc_feature (51)..(51): n is a or t
ataggtgtaagttggcatttatatatttgccagtttaaaaatacatcatangtaaggcaatgagaagagttttaaggacaattagtgataccttttgggtc

SEQ ID NO: 8 - DNA, 44 bp, Artificial sequence
  rs2303152 genotyping probe/primer
acttcgtcagtaacggacaaactttcaaaaagaaaaatgggggt

SEQ ID NO: 9 - DNA, 44 bp, Artificial sequence
  rs2303152 genotyping probe/primer
gagtcgaggtcatatcgtaaactttcaaaaagaaaaatgggggc

SEQ ID NO: 10 - DNA, 65 bp, Artificial sequence
  rs2303152 genotyping probe/primer
attccaaattccagtagctacaggaagccgctcttcttagtgatctggtctgcctatagtgagtc

SEQ ID NO: 11 - DNA, 45 bp, Artificial sequence
  rs12654264 genotyping probe/primer
acttcgtcagtaacggacagtaacctttcttttctgaagcatcct

SEQ ID NO: 12 - DNA, 45 bp, Artificial sequence
  rs12654264 genotyping probe/primer
gagtcgaggtcatatcgtagtaacctttcttttctgaagcatcca

SEQ ID NO: 13 - DNA, 68 bp, Artificial sequence
  rs12654264 genotyping probe/primer
tatatagagactgtgcatttttaatgttcaaaggtagacccgacacgtttgtctgcctatagtgagtc

SEQ ID NO: 14 - DNA, 42 bp, Artificial sequence
  rs12916 genotyping probe/primer
acttcgtcagtaacggacgtgcaattgaccttctccctcact

SEQ ID NO: 15 - DNA, 42 bp, Artificial sequence
  rs12916 genotyping probe/primer
gagtcgaggtcatatcgtgtgcaattgaccttctccctcacc

SEQ ID NO: 16 - DNA, 62 bp, Artificial sequence
  rs12916 genotyping probe/primer
ctgccagttgaaaatggatttgaatttacgcattgtgactggacgtctgcctatagtgagtc

SEQ ID NO: 17 - DNA, 47 bp, Artificial sequence
  rs4704209 genotyping probe/primer
acttcgtcagtaacggacccatgtaattccataatgtggctatctat

SEQ ID NO: 18 - DNA, 47 bp, Artificial sequence
  rs4704209 genotyping probe/primer
gagtcgaggtcatatcgtccatgtaattccataatgtggctatctac

SEQ ID NO: 19 - DNA, 66 bp, Artificial sequence
  rs4704209 genotyping probe/primer
gtctagaaactagaccataaaggaaatcagtcgtgcgatgttccaagcgtctgcctatagtgagtc

SEQ ID NO: 20 - DNA, 48 bp, Artificial sequence
  rs2241402 genotyping probe/primer
acttcgtcagtaacggacgccaaaattgtagaaaaaaagaaatcttat

SEQ ID NO: 21 - DNA, 48 bp, Artificial sequence
  rs2241402 genotyping probe/primer
gagtcgaggtcatatcgtgccaaaattgtagaaaaaaagaaatcttaa

SEQ ID NO: 22 - DNA, 65 bp, Artificial sequence
  rs2241402 genotyping probe/primer
aataatgagattggaactgaggaatcgggatggtcacaacatttcgtgtctgcctatagtgagtc

SEQ ID NO: 23 - DNA, 48 bp, Artificial sequence
  rs5908 genotyping probe/primer
acttcgtcagtaacggacgtgcacgtctacagaaacttcatacaagta

SEQ ID NO: 24 - DNA, 48 bp, Artificial sequence
  rs5908 genotyping probe/primer
gagtcgaggtcatatcgtgtgcacgtctacagaaacttcatacaagtg

SEQ ID NO: 25 - DNA, 61 bp, Artificial sequence
  rs5908 genotyping probe/primer
agctggacgcaacctttatattctccggctgatggaaccgtaggtctgcctatagtgagtc

SEQ ID NO: 26 - DNA, 48 bp, Artificial sequence
  rs10515198 genotyping probe/primer
acttcgtcagtaacggaccaattcttaaatcttgtgctatgaagaaat

SEQ ID NO: 27 - DNA, 48 bp, Artificial sequence
  rs10515198 genotyping probe/primer
gagtcgaggtcatatcgtcaattcttaaatcttgtgctatgaagaaac

SEQ ID NO: 28 - DNA, 68 bp, Artificial sequence
  rs10515198 genotyping probe/primer
ctattaatccttcctattaatgtaaagatgccaatatgacgattgctagagtctgcctatagtgagtc